(19) United States
(12) Patent Application Publication
(10) Pub. No.: US 2008/0045589 A1
(43) Pub. Date: Feb. 21, 2008

(54) Drug Combinations with Substituted Diaryl Ureas for the Treatment of Cancer

(76) Inventor: Susan Kelley, Woodbridge, CT (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/754,082

(22) Filed: May 25, 2007

Related U.S. Application Data
(60) Provisional application No. 60/808,555, filed on May 26, 2006.
(60) Provisional application No. 60/859,241, filed on Nov. 16, 2006.

(51) Int. Cl. A61K 31/282; A61P 35/00; C07D 213/81; C07D 305/14; C07F 15/00; C07K 14/76

(52) U.S. Cl. 514/492; 530/362; 546/298; 549/510; 556/137

(57) ABSTRACT

The present invention relates to drug combinations and pharmaceutical compositions for treating cancer such as non-small cell lung carcinoma, said combination comprising (1) at least one substituted-diaryl urea such as BAY 43-9006, (2) at least one taxane such as Paclitaxel (Taxol®), Docetaxel (Taxotere®) and Abraxane™ and (3) at least one platinum complex antineoplastic nucleic acid binding agent such as carboplatin (Paraplatin®), oxaplatin (Eloxatin®) and cisplatin (Platinol®), where any of these components can be present in the form of a pharmaceutically acceptable salt or other derivative thereof.

This application claims the benefit of the filing date of U.S. Provisional application No. 60/808,555, filed May 26, 2006 and U.S. Provisional application No. 60/859,241, filed Nov. 16, 2006, which are both incorporated by reference herein.

### FIELD OF THE INVENTION

This invention relates to drug combinations of substituted diaryl urea kinase inhibitors with two other chemotherapeutic agents and their use in treating hyper-proliferative disorders such as cancer, in humans and other mammals.

### BACKGROUND OF THE INVENTION

Non-small cell lung cancer (NSCLC) is a heterogeneous aggregate of at least three different histologies of lung cancer including epidermoid or squamous carcinoma, adenocarcinoma, and large cell carcinoma. They are often classified together because, in their localized states, all have the potential for cure with surgical procedure. At diagnosis, patients with NSCLC can be divided into three groups that reflect the extent of disease and treatment approach. The first group is characterized by surgically resectable tumors, and can be staged I or II. This is the group with the best prognosis, depending on a variety of tumor and host factors. The second group includes patients with advanced lung cancer and can be sub-categorized as local or regional. Radiation therapy with or without chemotherapy or other therapy modalities is the preferred mode of treatment. The final group comprises patients with distant metastasis. This group can be treated with radiation therapy or chemotherapy for palliation of symptoms from the primary tumor. Cisplatin-based chemotherapy has been associated with short-term palliation of symptoms and a small survival advantage.

For operable patients, prognosis is adversely influenced by the presence of pulmonary symptoms, large tumor size (>3 centimeters), and presence of the Erb-2 oncoprotein.[1-6] Other factors that have been identified as adverse prognostic factors in some series of patients with resectable non-small cell lung cancer include mutation of the K-ras gene, vascular invasion, and increased numbers of blood vessels in the tumor specimen.[3,7,8]

### REFERENCES

1. Albain K S, Crowley J J, LeBlanc M, et al.: Survival determinants in extensive-stage non-small-cell lung cancer: the Southwest Oncology Group experience. Journal of Clinical Oncology 9(9): 1618-1626, 1991.
2. Macchiarini P, Fontanini G, Hardin M J, et al.: Blood vessel invasion by tumor cells predicts recurrence in completely resected T1 N0 M0 non-small-cell lung cancer. Journal of Thoracic and Cardiovascular Surgery 106(1): 80-89, 1993.
3. Harpole D H, Herndon J E, Wolfe W G, et al.: A prognostic model of recurrence and death in stage I non-small cell lung cancer utilizing presentation, histopathology, and oncoprotein expression. Cancer Research 55(1): 51-56, 1995.
4. Ichinose Y, Yano T, Asoh H, et al.: Prognostic factors obtained by a pathologic examination in completely resected non-small-cell lung cancer: an analysis in each pathologic stage. Journal of Thoracic and Cardiovascular Surgery 110(3): 601-605, 1995.
5. Martini N, Bains M S, Burt M E, et al.: Incidence of local recurrence and second primary tumors in resected stage I lung cancer. Journal of Thoracic and Cardiovascular Surgery 109(1): 120-129, 1995.
6. Strauss G M, Kwiatkowski D J, Harpole D H, et al.: Molecular and pathologic markers in stage I non-small-cell carcinoma of the lung. Journal of Clinical Oncology 13(5): 1265-1279, 1995.
7. Slebos R J, Kibbelaar R E, Dalesio O, et al.: K-RAS oncogene activation as a prognostic marker in adenocarcinoma of the lung. New England Journal of Medicine 323(9): 561-565, 1990.
8. Fontanini G, Bigini D, Vignati S, et al.: Microvessel count predicts metastatic disease and survival in non-small cell lung cancer. Journal of Pathology 177: 57-63, 1995.

Prior to initiating treatment of any patient with lung cancer, a review of pathologic material by an experienced lung cancer pathologist can be important since the chemo-responsive small cell lung cancer can be confused with non-small cell carcinoma [1]. Histologic classification of non-small cell lung cancer can be squamous cell (epidermoid) carcinoma, adenocarcinoma, large cell carcinoma, adenosquamous carcinoma, and undifferentiated carcinoma. Similarly the staging procedure can be performed using the guidelines set by the American Joint Committee on Cancer (AJCC). Since the classification is based on characterization of the primary tumor (T), measurement of the size of lymph node (N), and assessment of distant metastasis (M), it is shortly known as TNM classification system for NSCLC.

In advanced-stage disease, chemotherapy has been reported to offer modest improvements in median survival.[1,2] Chemotherapy has been reported to produce short-term improvement in disease-related symptoms, while combination chemotherapy has been reported to yield symptomatic relief [3,4].

Several new agents, including paclitaxel (Taxol), docetaxel (Taxotere), topotecan, irinotecan, vinorelbine, and gemcitabine have been shown to be active in the treatment of advanced NSCLC.

### REFERENCES

1. Souquet P J, Chauvin F, Boissel J P, et al.: Polychemotherapy in advanced non small cell lung cancer: a meta-analysis. Lancet 342(8862): 19-21, 1993.
2. Non-small Cell Lung Cancer Collaborative Group: Chemotherapy in non-small cell lung cancer: a meta-analysis using updated data on individual patients from 52 randomised clinical trials. British Medical Journal 311(7010): 899-909, 1995.
3. Hardy J R, Noble T, Smith I E: Symptom relief with moderate dose chemotherapy (mitomycin-C, vinblastine and cisplatin) in advanced non-small cell lung cancer. British Journal of Cancer 60(5): 764-766, 1989.
4. Ellis P A, Smith I E, Hardy J R, et al.: Symptom relief with MVP (mitomycin C, vinblastine and cisplatin) chemotherapy in advanced non-small-cell lung cancer. British Journal of Cancer 71(2): 366-370, 1995.

Cisplatin-containing and carboplatin-containing combination chemotherapy regimens are reported to produce objective response rates (including a few complete responses) that are higher than those achieved with single-agent chemotherapy. Although toxic effects may vary, outcome is similar with most cisplatin-containing regimens; a randomized trial comparing 5 cisplatin-containing regimens reported no significant difference in response, duration of response, or survival.[1] Patients with good performance status and a limited number of sites of distant metastases were reported to have superior response and survival when given chemotherapy when compared to other patients.[2]. Two small phase II studies reported that paclitaxel (Taxol) has single-agent activity, with response rates in the range of 21% to 24%.[4,5] Reports of paclitaxel combinations have shown relatively high response rates, significant 1 year survival, and palliation of lung cancer symptoms.[6] With the paclitaxel plus carboplatin regimen, response rates have been in the range of 27% to 53% with 1-year survival rates of 32% to 54%.[6,7] The combination of cisplatin and paclitaxel was shown to have a higher response rate than the combination of cisplatin and etoposide.[8]

Although these results support further evaluation of chemotherapeutic approaches for both metastatic and locally advanced non-small cell lung cancer (NSCLC), efficacy of current programs, it is reported that no specific regimen can be regarded as standard therapy. Radiation therapy may be effective in palliating symptomatic local involvement with NSCLC such as tracheal, esophageal, or bronchial compression, bone or brain metastases, pain, vocal cord paralysis, hemoptysis, or superior vena cava syndrome.

The following Chemotherapy regimens have been associated with similar survival outcomes:
cisplatin plus vinblastine plus mitomycin [15]
cisplatin plus vinorelbine [3]
cisplatin plus paclitaxel [8]
cisplatin plus gemcitabine [16]
carboplatin plus paclitaxel [6,7]

### REFERENCES

1. Weick J K, Crowley J, Natale R B, et al.: A randomized trial of five cisplatin-containing treatments in patients with metastatic non-small-cell lung cancer: a Southwest Oncology Group study. Journal of Clinical Oncology 9(7): 1157-1162, 1991.
2. O'Connell J P, Kris M G, Gralla R J, et al.: Frequency and prognostic importance of pretreatment clinical characteristics in patients with advanced non-small-cell lung cancer treated with combination chemotherapy. Journal of Clinical Oncology 4(11): 1604-1614, 1986.
3. Le Chevalier T, Brisgand D, Douillard J Y, et al.: Randomized study of vinorelbine and cisplatin versus vindesine and cisplatin versus vinorelbine alone in advanced non-small-cell lung cancer: results of a European multicenter trial including 612 patients. Journal of Clinical Oncology 12(2): 360-367, 1994.
4. Chang A Y, Kim K, Glick J, et al.: Phase II study of taxol, merbarone, and piroxantrone in stage IV non-small-cell lung cancer: the Eastern Cooperative Oncology Group results. Journal of the National Cancer Institute 85(5): 388-394, 1993.
5. Murphy W K, Fossella F V, Winn R J, et al.: Phase II study of taxol in patients with untreated advanced non-small-cell lung cancer. Journal of the National Cancer Institute 85(5): 384-388, 1993.
6. Johnson D H, Paul D M, Hande K R, et al.: Paclitaxel plus carboplatin in advanced non-small-cell lung cancer: a phase II trial. Journal of Clinical Oncology 14(7): 2054-2060, 1996.
7. Langer C J, Leighton J C, Comis R L, et al.: Paclitaxel and carboplatin in combination in the treatment of advanced non-small-cell lung cancer: a phase II toxicity, response, and survival analysis. Journal of Clinical Oncology 13(8): 1860-1870, 1995.
8. Bonomi P, Kim K, Chang A, et al.: Phase III trial comparing etoposide (E) cisplatin (C) versus taxol (T) with cisplatin-G-CSF (G) versus taxol-cisplatin in advanced non-small cell lung cancer. An Eastern Cooperative Oncology Group (ECOG) trial. Proceedings of the American Society of Clinical Oncology 15: A-1145, 382, 1996.
9. Souquet P J, Chauvin F, Boissel J P, et al.: Polychemotherapy in advanced non small cell lung cancer: a meta-analysis. Lancet 342(8862): 19-21, 1993.

The lung is also frequently the site of second primary malignancies in patients with primary lung cancers. Determining whether the new lesion is a new primary cancer or a metastasis may be difficult. Studies have indicated that in the majority of patients the new lesion is a second primary tumor, and following resection some patients may achieve long-term survival. Thus, if the first primary tumor has been controlled, the second primary tumor should be resected if possible.[8,9] It is reported that the use of chemotherapy has produced objective responses and small improvement in survival for patients with metastatic disease.[10]

Treatment Options:

1. Palliative radiation therapy.

2. Chemotherapy alone. For patients who have not received prior chemotherapy, the following regimens are associated with similar survival outcomes:

cisplatin plus vinblastine plus mitomycin [13]
cisplatin plus vinorelbine [14]
cisplatin plus paclitaxel [15]
cisplatin plus gemcitabine [16]
carboplatin plus paclitaxel [17,18]
   3. Surgical resection of isolated cerebral metastasis (highly selected patients).[6]
   4. Laser therapy or interstitial radiation therapy for endobronchial lesions.[19]
   5. Stereotactic radiosurgery (highly selected patients).[3,5]

### REFERENCES

1. Patchell R A, Tibbs P A, Walsh J W, et al.: A randomized trial of surgery in the treatment of single metastases to the brain. New England Journal of Medicine 322(8): 494-500, 1990.
2. Mandell L, Hilaris B, Sullivan M, et al.: The treatment of single brain metastasis from non-oat cell lung carcinoma: surgery and radiation versus radiation therapy alone. Cancer 58(3): 641-649, 1986.
3. Loeffler J S, Kooy H M, Wen P Y, et al.: The treatment of recurrent brain metastases with stereotactic radiosurgery. Journal of Clinical Oncology 8(4): 576-582, 1990.
4. DeAngelis L M, Mandell L R, Thaler H T, et al.: The role of postoperative radiotherapy after resection of single brain metastases. Neurosurgery 24(6): 798-805, 1989.
5. Alexander E, Moriarty T M, Davis R B, et al.: Stereotactic radiosurgery for the definitive, noninvasive treatment of brain metastases. Journal of the National Cancer Institute 87(1): 34-40, 1995.
6. Arbit E, Wronski M, Burt M, et al.: The treatment of patients with recurrent brain metastases: a retrospective analysis of 109 patients with nonsmall cell lung cancer. Cancer 76(5): 765-773, 1995.
7. Hazuka M B, Kinzie J J: Brain metastases: results and effects of re-irradiation. International Journal of Radiation Oncology, Biology, Physics 15(2): 433-437, 1988.
8. Salerno T A, Munro D D, Blundell P E, et al.: Second primary bronchogenic carcinoma: life-table analysis of surgical treatment. Annals of Thoracic Surgery 27(1): 3-6, 1979.
9. Yellin A, Hill L R, Benfield J R: Bronchogenic carcinoma associated with upper aerodigestive cancer. Journal of Thoracic and Cardiovascular Surgery 91(5): 674-683, 1986.
10. Souquet P J, Chauvin F, Boissel J P, et al.: Polychemotherapy in advanced non small cell lung cancer: a meta-analysis. Lancet 342(8862): 19-21, 1993.
11. Ellis P A, Smith I E, Hardy J R, et al.: Symptom relief with MVP (mitomycin C, vinblastine and cisplatin) chemotherapy in advanced non-small-cell lung cancer. British Journal of Cancer 71(2): 366-370, 1995.
12. Medical Research Council Lung Cancer Working Party: Randomized trial of etoposide cyclophosphamide methotrexate and vincristine versus etoposide and vincristine in the palliative treatment of patients with small-cell lung cancer and poor prognosis. British Journal of Cancer 67(Suppl 20): A-4; 2, 14, 1993.
13. Veeder M H, Jett J R, Su J Q, et al.: A phase III trial of mitomycin C alone versus mitomycin C, vinblastine, and cisplatin for metastatic squamous cell lung carcinoma. Cancer 70(9): 2281-2287, 1992.
14. Le Chevalier T, Brisgand D, Douillard J Y, et al.: Randomized study of vinorelbine and cisplatin versus vindesine and cisplatin versus vinorelbine alone in advanced non-small-cell lung cancer: results of a European multicenter trial including 612 patients. Journal of Clinical Oncology 12(2): 360-367, 1994.
15. Bonomi P, Kim K, Chang A, et al.: Phase III trial comparing etoposide (E) cisplatin (C) versus taxol (T) with cisplatin-G-CSF (G) versus taxol-cisplatin in advanced non-small cell lung cancer. An Eastern Cooperative Oncology Group (ECOG) trial. Proceedings of the American Society of Clinical Oncology 15: A-1145, 382, 1996.
16. Rosell R, Tonato M, Sandier A: The activity of gemcitabine plus cisplatin in randomized trials in untreated patients with advanced non-small cell lung cancer. Seminars in Oncology 25(4 suppl 9): 27-34, 1998.
17. Johnson D H, Paul D M, Hande K R, et al.: Paclitaxel plus carboplatin in advanced non-small-cell lung cancer: a phase II trial. Journal of Clinical Oncology 14(7): 2054-2060, 1996.
18. Langer C J, Leighton J C, Comis R L, et al.: Paclitaxel and carboplatin in combination in the treatment of advanced non-small-cell lung cancer: a phase II toxicity, response, and survival analysis. Journal of Clinical Oncology 13(8): 1860-1870, 1995.
19. Miller J I, Phillips T W: Neodymium:YAG laser and brachytherapy in the management of inoperable bronchogenic carcinoma. Annals of Thoracic Surgery 50(2): 190-196, 1990.

Substituted Diarylureas are a class of serine-threonine kinase inhibitors as well as tyrosine kinase inhibitors known in the art (Smith et al., Bioorg. Med. Chem. Lett. 2001, 11, 2775-2778, Lowinger et al., Clin. Cancer Res. 2000, 6(suppl.), 335, Lyons et al., Endocr.-Relat. Cancer 2001, 8, 219-225, Lowinger et al., Curr. Pharm. Design 2002, 8, 99-110). Omega-Carboxyaryl diphenyl ureas are disclosed in WO00/42012 and WO00/41698. In particular, it has been discovered that the diphenyl urea of formula (A), 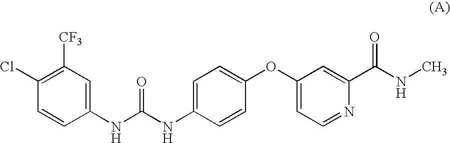
also referred as “BAY 43-9006” or “4-{4-[({[4-chloro-3-(trifluoromethyl)phenyl]amino}carbonyl)amino]phenoxy}-N-methylpyridine-2-carboxamide”, or “N-(4-chloro-3-(trifluoromethyl)phenyl-N′-(4-(2-(N-methylcarbamoyl)-4-pyridyloxy)phenyl)urea”, or “4 {4-[3-(4-chloro-3-trifluoromethylphenyl)-ureido]-phenoxy}-pyridine-2-carboxylic acid methyl amide” and its pharmaceutically acceptable salts are potent inhibitors of raf, VEGFR-2, p38, PDGFR and/or flt-3 signaling kinases. See, e.g., US 20050038080. These enzymes are all molecular targets of interest for the treatment of hyper-proliferative diseases, including cancer.

Combinations of Sorafenib (a drug which contains the tosylate salt of BAY 43-9006 as an active ingredient) with carboplatin and paclitaxel are reported to be undergoing Phase III clinical trials for treating Unresectable Stage III or Stage IV Melanoma and Recurrent Ovarian Cancer, Primary Peritoneal Cancer and Fallopian Tube Cancer. (See www.clinicaltrials.gov.

The use of the substituted diaryl urea, BAY 43-9006, in treating non-small cell lung cancer has been disclosed. See Lee and MeCubrey, Curr. Opin. Investig. Drugs, 4:657-63, June 2003 and Wilhelm et al., Cancer Res., 64:7099-7109, 2004, WO/04096224 (Boehringer; Hilberg et al.) published Nov. 11, 2004; Apr. 29, 2003.

WO 03/047579 relates to the use of substituted diaryl ureas in combination with cytotoxic or cytostatic compounds for treating cancer.

Methods for preparing substituted diaryl ureas such as BAY 43-9006 are described in the following U.S. application Ser. Nos.:

09/425,228, filed Oct. 22, 1999;

09/722,418 filed Nov. 28, 2000

09/758,547, filed Jan. 12, 2001;

09/838,285, filed Apr. 20, 2001;

09/838,286, filed Apr. 20, 2001; and

### DESCRIPTION OF THE INVENTION

The present invention provides drug combinations, pharmaceutical compositions, and methods for treating diseases and conditions, including, but not limited to, cell proliferative disorders such as cancer, including but not limited to colon, gastric, lung, pancreatic, ovarian, prostate, leukemia, melanoma, hepatocellular, renal, head and neck, glioma, and mammary cancers.

The drug combinations comprise (1) at least one substituted-diaryl urea of Formula I (defined below), (2) at least one taxane such as Paclitaxel (Taxol®), Docetaxel (Taxotere®) and Abraxane™ and (3) at least one platinum complex antineoplastic nucleic acid binding agent such as carboplatin (Paraplatin®), oxaplatin (Eloxatin®) and cisplatin (Platinol®), where any of these components can be present in the form of a pharmaceutically acceptable salt or other known derivative.

In a preferred embodiment of the drug combination, the substituted diaryl urea is Sorafenib, the taxane is paclitaxel and the platinum complex is carboplatin.

The invention also relates to pharmaceutical compositions which comprise one or more pharmaceutically acceptable carrier molecules and quantities of substituted diaryl urea compound of Formula I (defined below), a taxane (e.g., paclitaxel) and a platinum complex (e.g., carboplatin), in amounts which are jointly effective for treating a cancer, where any of these components can be present in the form of a pharmaceutically acceptable salt or other common derivative.

The methods can comprise, e.g., administering (1) a substituted diaryl urea compound of Formula I (e.g. Sorafenib); (2) a taxane (e.g. paclitaxel) and (3) a platinum complex (e.g., carboplatin) or pharmaceutically-acceptable salts or derivatives thereof, etc.

In particular embodiments of this invention, the active components of the drug combination are administered to a patient by oral delivery and/or by intravenous injection or infusion.

In particular embodiments of this invention, the substituted diaryl urea compound of Formula I is administered simultaneously with the taxane (e.g. paclitaxel) and platinum complex (e.g. carboplatin) to a patient with cancer, in the same formulation or in separate formulations, optionally using different administration routes. Administration can also be sequentially, in any order.

In particular embodiments of this invention, the substituted diaryl urea compounds of Formula I (e.g., Sorafenib) are administered in tandem with the taxane (e.g., paclitaxel) and platinum complex (e.g., carboplatin), wherein the substituted diaryl urea compound of Formula I is administered to a patient once or more per day for up to 28 consecutive days with the concurrent or intermittent administration of the taxane and platinum complex over the same total time period.

In particular embodiments of this invention, the substituted diaryl urea compound of Formula I (e.g., Sorafenib) can be administered to a patient as an oral, intravenous, intramuscular, subcutaneous, or parenteral dosage which can range from about 0.1 to about 300 mg/kg of total body weight.

In particular embodiments of this invention, the taxane (e.g., paclitaxel) is administered to a patient as an intravenous, intramuscular, subcutaneous, or parenteral dosage which can range from about 10-300 mg/m² of patient surface area.

In particular embodiments of this invention, the platinum complex (e.g., carboplatin) is administered to a patient at an intravenous, intramuscular, subcutaneous, or parenteral dosage which can range from about 100-500 mg/m² of patient surface area.

In particular embodiments of this invention, the substituted diaryl urea compound of Formula I is Sorafenib, a tosylate salt of N-(4-chloro-3-(trifluoromethyl)phenyl)-N′-(4-(2-(N-methylcarbamoyl)-4-pyridyloxy)phenyl)urea. The scaleable synthesis of the aryl urea compound is disclosed in Organic Process Research and Development (2002), Vol. 6, Issue #6, 777-781, and copending patent application Ser. No. 09/948,915 filed Sep. 10, 2001 which are incorporated herein by reference.

This invention also relates to compositions containing a substituted diaryl urea compound of formula I, paclitaxel and carboplatin in amounts consistent with the objectives of this invention. This invention further relates to kits comprising separate doses of the three chemotherapeutic agents in separate containers. The combinations of the invention can also be formed in vivo, e.g., in a patient's body.

Paclitaxel is sold under the tradename Taxol® by the Bristol-Myers Squibb Company. Paclitaxel (5∃,20-Epoxy-1,2∀,4,7∃,10∃,13∀-hexahydroxytax-11-en-9-one 4,10-diacetate 2-benzoate 13-ester with (2R,3S)—N-benzoyl-3-phenylisoserine) has the empirical formula C₄₇H₅₁NO₁₄ and a molecular weight of 853.9. It is highly lipophilic in water. Paclitaxel is an antimicrotubule agent that promotes the assembly of microtubles from tubulin dimers and stabilizes microtubules by preventing depolymerization. While not bound by a theory, it is believed that this stability results in the inhibition in the normal dynamic reorganization of the microtubule network that is essential for vital interphase and mitotic cellular functions. Also, paclitaxel is believed to induce abnormal arrays or bundles of microtubules throughout the cell cycle and multiple asters of microtubules during mitosis. Paclitaxel is administered by intravenous injection or by other appropriate infusion techniques in the conventional formulations and regimens in which they are known for use alone.

The substituted diaryl urea compounds of formula (I), salts, polymorphs, solvates, hydrates metabolites and prodrugs thereof, including diastereoisomeric forms (both isolated stereoisomers and mixtures of stereoisomers) are collectively referred to herein as the “Compounds of Formula I”. Formula (I) is as follows: 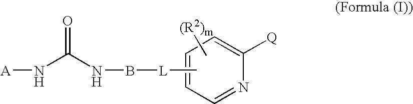
wherein
Q is —C(O)R)ₓ
Rₓ is hydroxy, C₁₋₄ alkyl, C₁₋₄ alkoxy or NRₐR_{b},
R^{a} and R^{b} are independently:
a) hydrogen;
b) C₁₋₄ alkyl, optionally substituted by
   hydroxy,
   C₁₋₄ alkoxy,
   a heteroaryl group selected from pyrrole, furan, thiophene, imidazole, pyrazole, thiazole, oxazole, isoxazole, isothiazole, triazole, tetrazole, thiadiazole, oxadiazole, pyridine, pyrimidine, pyridazine, pyrazine, triazine, benzoxazole, isoquioline, quinolines and imidazopyrimidine
   a heterocyclic group selected from tetrahydropyran, tetrahydrofuran, 1,3-dioxolane, 1,4-dioxane, morpholine, thiomorpholine, piperazine, piperidine, piperidinone, tetrahydropyrimidone, pentamethylene sulfide, tetramethylene sulfide, dihydropyrane, dihydrofuran, and dihydrothiophene,
   amino, —NH₂, optionally substituted by one or two C₁₋₄ alkyl groups, or
   phenyl,
c) phenyl optionally substituted with
   halogen, or
   amino, —NH₂, optionally substituted by one or two C₁₋₄ alkyl, or
d) a heteroaryl group selected from pyrrole, furan, thiophene, imidazole, pyrazole, thiazole, oxazole, isoxazole, isothiazole, triazole, tetrazole, thiadiazole, oxadiazole, pyridine, pyrimidine, pyridazine, pyrazine, triazine, benzoxazole, isoquioline, quinoline and imidazopyrimidine;
A is optionally substituted phenyl, pyridinyl, naphthyl, benzoxazole, isoquioline, quinoline or imidazopyrimidine;
B is optionally substituted phenyl or naphthyl:
L is a bridging group which is —S— or —O—;
M is 0,1,2 or 3, and
each R² is independently C₁₋₅ alkyl, C₁₋₅haloalkyl, C₁₋₃alkoxy, N-oxo or N-hydroxy.

Structures of optionally substituted phenyl moieties for A of formula (I) which are of particular interest include structures of formula 1xx:

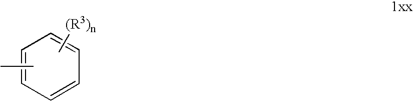

Structures of optionally substituted pyridinyl moieties for A of formula (I) which are of particular interest include structures of formula 1x:

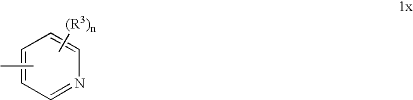

Structures of optionally substituted naphthyl moieties for A of formula (I) which are of particular interest include structures of formula 1y:

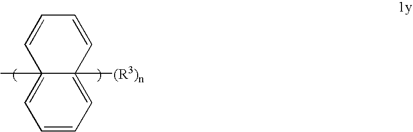

The structure 1y represents that the substituents R³ can appear on any carbon atom in either ring which has a valence that is otherwise complete with a hydrogen atom as a substituent. The bond to the urea group can also be through any carbon atom on either ring which has a valence that is otherwise complete with a hydrogen atom as a substituent.

B is optionally substituted phenyl or naphthyl. Structures of optionally substituted phenyl or naphthyl moieties for B of formula (I) which are of particular interest include structures 2a and 2b:

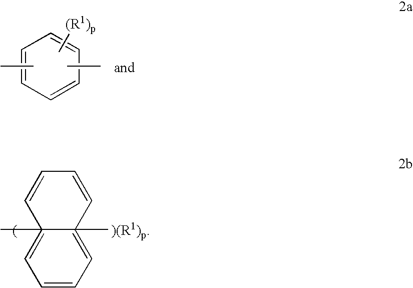

The structures 2a and 2b represent that the substituents R¹ can appear on any carbon atom in the structure which has a valence that is otherwise complete with a hydrogen atom as a substituent and the bond to the urea group can be through any carbon atom in the structure which has a valence that is otherwise complete with a hydrogen atom as a substituent.

In a class of embodiments of this invention, B is substituted by at least one halogen substituent. In another class of embodiments, Rₓ is NRₐR_{b}, and Rₐ and R_{b} are independently hydrogen or C₁₋₄ alkyl optionally substituted by hydroxy and L is a bridging group which is —S— or —O—.

The variable p is 0, 1, 2, 3, or 4, typically 0 or 1. The variable n is 0, 1, 2, 3, 4, 5 or 6, typically 0, 1, 2, 3 or 4. The variable m is 0, 1, 2 or 3, typically 0.

Each R¹ is independently: halogen, C₁₋₅ haloalkyl, NO₂, C(O)NR⁴R⁵, C₁₋₆ alkyl, C₁₋₆ dialkylamine, C₁₋₃ alkylamine, CN, amino, hydroxy or C₁₋₃ alkoxy. Where present, R¹ is more commonly halogen and of the halogens, typically chlorine or fluorine, and more commonly fluorine.

Each R² is independently: C₁₋₅ alkyl, C₁₋₅ haloalkyl, C₁₋₃ alkoxy, N-oxo or N-hydroxy. Where present, R² is typically methyl or trifluoromethyl.

Each R³ is independently selected from halogen, R⁴, OR⁴, S(O)R⁴, C(O)R⁴, C(O)NR⁴R⁵, oxo, cyano or nitro (NO₂).

R⁴ and R⁵ are independently selected from hydrogen, C₁₋₆ alkyl, and up to per-halogenated C₁₋₆ alkyl.

Other examples of A include: 3-tert butyl phenyl, S-tert butyl-2-methoxyphenyl, 5-(trifluoromethyl)-2 phenyl, 3-(trifluoromethyl)-4-chlorophenyl, 3-(trifluoromethyl)-4-bromophenyl and 5-(trifluoromethyl)-4-chloro-2 methoxyphenyl.

Other examples of B include:

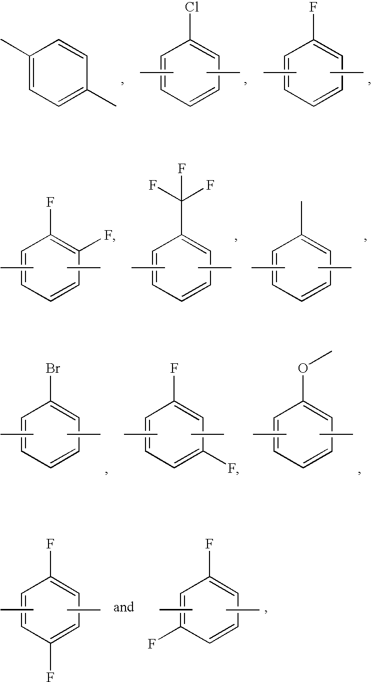

Preferably the urea group —NH—C(O)—NH— and the bridging group, L, are not bound to contiguous ring carbons of B, but rather have 1 or 2 ring carbons separating them.

Examples of R¹ groups include fluorine, chorine, bromine, methyl, NO₂, C(O)NH₂, methoxy, SCH₃, trifluoromethyl, and methanesulfonyl.

Examples of R² groups include methyl, ethyl, propyl, oxygen, and cyano.

Examples of R³ groups include trifluoromethyl, methyl, ethyl, propyl, butyl, isopropyl, tert-butyl, chlorine, fluorine, bromine, cyano, methoxy, acetyl, trifluoromethanesulfonyl, trifluoromethoxy, and trifluoromethylthio.

A class of compounds of interest are of formula II below 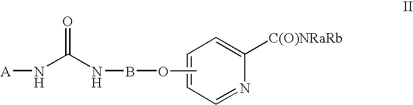
wherein Ra and Rb are independently hydrogen and C₁-C₄ alkyl, B of formula II is

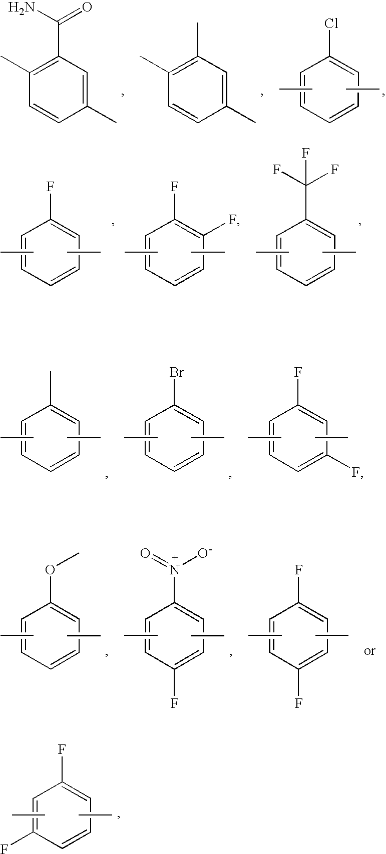

wherein the urea group, —NH—C(O)—NH—, and the oxygen bridging group are not bound to contiguous ring carbons of B, but rather have 1 or 2 ring carbons separating them,
and A of formula (II) is 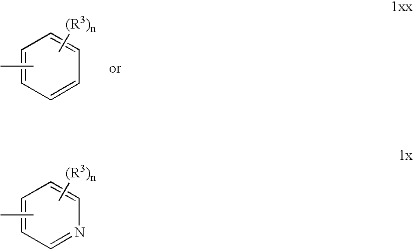
wherein the variable n is 0, 1, 2, 3 or 4.

R³ is trifluoromethyl, methyl, ethyl, propyl, butyl, isopropyl, tert-butyl, chlorine, fluorine, bromine, cyano, methoxy, acetyl, trifluoromethanesulfonyl, trifluoromethoxy, or trifluoromethylthio.

In a subclass of such compounds, each R³ substituent on A of formula II is selected from chlorine, trifluoromethyl, tert-butyl or methoxy.

In another subclass of such compounds, A of formula II is 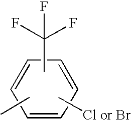
and B of formula II is phenylene, fluoro substituted phenylene or difluoro substituted phenylene.

Another class of compounds of interest includes compounds having the structure of formulae X below wherein phenyl ring “B” optionally has one halogen substituent, preferably Cl or F, more preferably F.

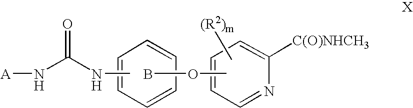

For the compounds of formula X, R², m and A are as defined above for formula I. The variable “m” is preferably zero, leaving C(O)NHCH₃ as the only substituent on the pyridinyl moiety. Preferred values for A are substituted phenyl which have at least one substituent, R³. R³ is preferably halogen, preferably Cl or F, trifluoromethyl and/or methoxy.

A subclass of compounds of interest includes compounds having the structure of formulas Z1, Z2, and Z3 below: 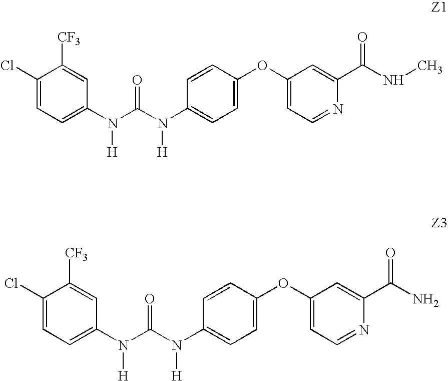
The compound Z1 is described in WO 00/42012. Both WO 00/42012 and Bankston et al. (Organic Process Research & Development, 2002, 6, 777-781) describe a process for preparing compound Z1, which is illustrated in the following scheme:

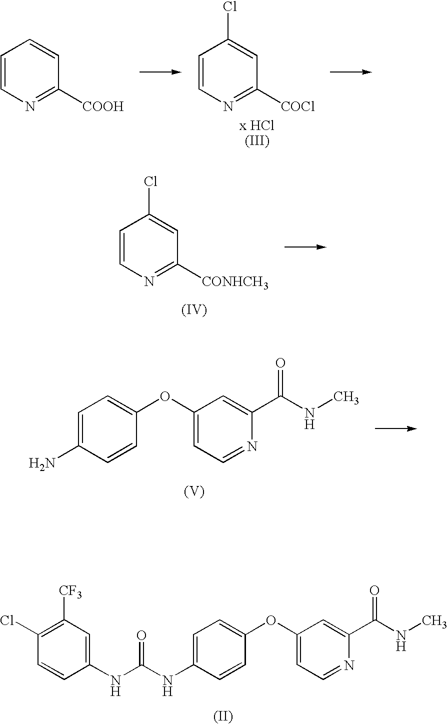

Preferably used in the pharmaceutical composition according to the invention is the p-toluenesulfonic acid salt of 4{4-[3-(4-chloro-3-trifluoromethylphenyl)-ureido]-phenoxy}-pyridine-2-carboxylic acid methyl amide (tosylate salt of compound (I)). More preferably the p-toluenesulfonic acid salt of 4{4-[3-(4-chloro-3-trifluoromethylphenyl)-ureido]-phenoxy}-pyridine-2-carboxylic acid methyl amide exists for at least 80% in the stable polymorph I. Most preferably the p-toluenesulfonic acid salt of 4{4-[3-(4-chloro-3-trifluoromethylphenyl)-ureido]-phenoxy}-pyridine-2-carboxylic acid methyl amide exists for at least 80% in the stable polymorph I and in a micronized form.

Micronization can be achieved by standard milling methods, preferably by air chat milling, known to a skilled person. The micronized form can have a mean particle size of from 0.5 to 10 μm, preferably from 1 to 6 μm, more preferably from 1 to 3 μm. The indicated particle size is the mean of the particle size distribution measured by laser diffraction known to a skilled person (measuring device: HELOS, Sympatec).

The process for preparing the p-toluenesulfonic acid salt of 4{4-[3-(4-chloro-3-trifluoromethylphenyl)-ureido]-phenoxy}-pyridine-2-carboxylic acid methyl amide and its stable polymorph I are described in the patent applications EP 04023131.8 and EP 04023130.0.

When any moiety is “substituted”, it can have up to the highest number of indicated substituents and each substituent can be located at any available position on the moiety and can be attached through any available atom on the substituent. “Any available position” means any position on the moiety that is chemically accessible through means known in the art or taught herein and that does not create an unstable molecule, e.g., incapable of administration to a human. When there are two or more substituents on any moiety, each substituent is defined independently of any other substituent and can, accordingly, be the same or different.

The term “optionally substituted” means that the moiety so modified may be either unsubstituted, or substituted with the identified substituent(s).

It is understood that the term “hydroxy” as a pyridine substituent includes 2-, 3-, and 4-hydroxypyridine, and also includes those structures referred to in the art as 1-oxo-pyridine, 1-hydroxy-pyridine or pyridine N-oxide.

Where the plural form of the word compounds, salts, and the like, is used herein, this is taken to mean also a single compound, salt, or the like.

The term C₁₋₆ alkyl, unless indicated otherwise, means straight, branched chain or cyclic alkyl groups having from one to six carbon atoms, which may be cyclic, linear or branched with single or multiple branching. Such groups include for example methyl, ethyl, n-propyl, isopropyl, n-butyl, isobutyl, sec-butyl, tert-butyl, cyclopropyl, cyclobutyl and the like.

The term C₁₋₆ haloalkyl, unless indicated otherwise, means a saturated hydrocarbon radical having up to six carbon atoms, which is substituted with a least one halogen atom, up to perhalo. The radical may be cyclic, linear or branched with single or multiple branching. The halo substituent(s) include fluoro, chloro, bromo, or iodo. Fluoro, chloro and bromo are preferred, and fluoro and chloro are more preferred. The halogen substituent(s) can be located on any available carbon. When more than one halogen substituent is present on this moiety, they may be the same or different. Examples of such halogenated alkyl substituents include but are not limited to chloromethyl, dichloromethyl, trichloromethyl, fluoromethyl, difluoromethyl, trifluoromethyl, 2,2,2-trifluoroethyl, and 1,1,2,2-tetrafluoroethyl, and the like.

The term C₁₋₆ alkoxy, unless indicated otherwise, means a cyclic, straight or branched chain alkoxy group having from one to six saturated carbon atoms which may be cyclic, linear or branched with single or multiple branching, and includes such groups as methoxy, ethoxy, n-propoxy, isopropoxy, butoxy, pentoxy and the like. It also includes halogenated groups such as 2,2-dichloroethoxy, trifluoromethoxy, and the like.

Halo or halogen means fluoro, chloro, bromo, or iodo. Fluoro, chloro and bromo are preferred, and fluoro and chloro are more preferred.

C₁₋₃alkylamine, unless indicated otherwise, means methylamino, ethylamino, propylamino or isopropylamino.

Examples of C₁₋₆ dialkylamine include but are not limited to diethylamino, ethyl-isopropylamino, methyl-isobutylamino and dihexylamino.

The term heteroaryl refers to both monocyclic and bicyclic heteroaryl rings. Monocyclic heteroaryl means an aromatic monocyclic ring having 5 to 6 ring atoms and 1-4 hetero atoms selected from N, O and S, the remaining atoms being carbon. When more than one hetero atom is present in the moiety, they are selected independently from the other(s) so that they may be the same or different. Monocyclic heteroaryl rings include, but are not limited to pyrrole, furan, thiophene, imidazole, pyrazole, thiazole, oxazole, isoxazole, isothiazole, triazole, tetrazole, thiadiazole, oxadiazole, pyridine, pyrimidine, pyridazine, pyrazine, and triazine.

Bicyclic heteroaryl means fused bicyclic moieties where one of the rings is chosen from the monocyclic heteroaryl rings described above and the second ring is either benzene or another monocyclic heteroaryl ring described above. When both rings in the bicyclic moiety are heteroaryl rings, they may be the same or different, as long as they are chemically accessible by means known in the art. Bicyclic heteroaryl rings include synthetically accessible 5-5, 5-6, or 6-6 fused bicyclic aromatic structures including, for example but not by way of limitation, benzoxazole (fused phenyl and oxazole), quinoline (fused phenyl and pyridine), imidazopyrimidine (fused imidazole and pyrimidine), and the like.

Where indicated, the bicyclic heteroaryl moieties may be partially saturated. When partially saturated either the monocyclic heteroaryl ring as described above is fully or partially saturated, the second ring as described above is either fully or partially saturated or both rings are partially saturated. The term “5 or 6 membered heterocyclic ring, containing at least one atom selected from oxygen, nitrogen and sulfur, which is saturated, partially saturated, or aromatic” includes, by no way of limitation, tetrahydropyran, tetrahydrofuran, 1,3-dioxolane, 1,4-dioxane, morpholine, thiomorpholine, piperazine, piperidine, piperidinone, tetrahydropyrimidone, pentamethylene sulfide, tetramethylene sulfide, dihydropyrane, dihydrofuran, dihydrothiophene, pyrrole, furan, thiophene, imidazole, pyrazole, thiazole, oxazole, isoxazole, isothiazole, triazole, pyridine, pyrimidine, pyridazine, pyrazine, triazine, and the like.

The term “C₁₋₃ alkyl-phenyl” includes, for example, 2-methylphenyl, isopropylphenyl, 3-phenylpropyl, or 2-phenyl-1-methylethyl. Substituted examples include 2-[2-chlorophenyl]ethyl, 3,4-dimethylphenylmethyl, and the like.

Unless otherwise stated or indicated, the term “aryl” includes 6-12 membered mono or bicyclic aromatic hydrocarbon groups (e.g., phenyl, naphthalene, azulene, indene group) having 0, 1, 2, 3, 4, 5 or 6 substituents.

The compounds of Formula (I) may contain one or more asymmetric centers, depending upon the location and nature of the various substituents desired. Asymmetric carbon atoms may be present in the (R) or (S) configuration or (R,S) configuration. In certain instances, asymmetry may also be present due to restricted rotation about a given bond, for example, the central bond adjoining two substituted aromatic rings of the specified compounds. Substituents on a ring may also be present in either cis or trans form. It is intended that all such configurations (including enantiomers and diastereomers), are included within the scope of the present invention. Preferred compounds are those with the absolute configuration of the compound of Formula (I) which produces the more desirable biological activity. Separated, pure or partially purified isomers or racemic mixtures of the compounds of this invention are also included within the scope of the present invention. The purification of said isomers and the separation of said isomeric mixtures can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallization. The optically active bases or acids are then liberated from the separated diastereomeric salts. A different process for separation of optical isomers involves the use of chiral chromatography (e.g., chiral HPLC columns), with or without conventional derivation, optimally chosen to maximize the separation of the enantiomers. Suitable chiral HPLC columns are manufactured by Diacel, e.g., Chiracel OD and Chiracel OJ among many others, all routinely selectable. Enzymatic separations, with or without derivitization, are also useful. The optically active compounds of Formula I can likewise be obtained by chiral syntheses utilizing optically active starting materials.

The present invention also relates to useful forms of the compounds as disclosed herein, such as pharmaceutically acceptable salts, metabolites and prodrugs of all the compounds Formula (I). The term “pharmaceutically acceptable salt” refers to a relatively non-toxic, inorganic or organic acid addition salt of a compound of the present invention. For example, see S. M. Berge, et al. “Pharmaceutical Salts,” J. Pharm. Sci. 1977, 66, 1-19. Pharmaceutically acceptable salts include those obtained by reacting the main compound, functioning as a base, with an inorganic or organic acid to form a salt, for example, salts of hydrochloric acid, sulfuric acid, phosphoric acid, methane sulfonic acid, camphor sulfonic acid, oxalic acid, maleic acid, succinic acid and citric acid. Pharmaceutically acceptable salts also include those in which the main compound functions as an acid and is reacted with an appropriate base to form, e.g., sodium, potassium, calcium, mangnesium, ammonium, and choline salts. Those skilled in the art will further recognize that acid addition salts of the claimed compounds may be prepared by reaction of the compounds with the appropriate inorganic or organic acid via any of a number of known methods. Alternatively, alkali and alkaline earth metal salts are prepared by reacting the compounds of the invention with the appropriate base via a variety of known methods.

Representative salts of the compounds of this invention include the conventional non-toxic salts and the quaternary ammonium salts which are formed, for example, from inorganic or organic acids or bases by means well known in the art. For example, such acid addition salts include acetate, adipate, alginate, ascorbate, aspartate, benzoate, benzenesulfonate, bisulfate, butyrate, citrate, camphorate, camphorsulfonate, cinnamate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, itaconate, lactate, maleate, mandelate, methanesulfonate, 2-naphthalenesulfonate, nicotinate, nitrate, oxalate, pamoate, pectinate, persulfate, 3-phenylpropionate, picrate, pivalate, propionate, succinate, sulfonate, tartrate, thiocyanate, tosylate, trifluoromethanesulfonate, and undecanoate.

Base salts include alkali metal salts such as potassium and sodium salts, alkaline earth metal salts such as calcium and magnesium salts, and ammonium salts with organic bases such as dicyclohexylamine and N-methyl-D-glucamine. Additionally, basic nitrogen containing groups may be quaternized with such agents as lower alkyl halides such as methyl, ethyl, propyl, and butyl chlorides, bromides and iodides; dialkyl sulfates like dimethyl, diethyl, and dibutyl sulfate; and diamyl sulfates, long chain halides such as decyl, lauryl, myristyl and strearyl chlorides, bromides and iodides, aryl or aralkyl halides like benzyl and phenethyl bromides and others monosubstituted aralkyl halides or polysubstituted aralkyl halides.

Solvates for the purposes of the invention are those forms of the compounds where solvent molecules form a complex in the solid state and include, but are not limited to for example ethanol and methanol. Hydrates are a specific form of solvates, where the solvent molecule is water.

Certain pharmacologically active agents can be further modified with labile functional groups that are cleaved after in vivo administration to furnish the parent active agent and the pharmacologically inactive derivatizing group. These derivatives, commonly referred to as prodrugs, can be used, for example, to alter the physicochemical properties of the active agent, to target the active agent to a specific tissue, to alter the pharmacokinetic and pharmacodynamic properties of the active agent, and to reduce undesirable side effects. Prodrugs of the invention include, e.g., the esters of appropriate compounds of this invention that are well-tolerated, pharmaceutically acceptable esters such as alkyl esters including methyl, ethyl, propyl, isopropyl, butyl, isobutyl or pentyl esters. Additional esters such as phenyl-C₁-C₅ alkyl may be used, although methyl ester is preferred.

Methods which can be used to synthesize other prodrugs are described in the following reviews on the subject, which are incorporated herein by reference for their description of these synthesis methods:
Higuchi, T.; Stella, V. eds. Prodrugs As Novel Drug Delivery Systems. ACS Symposium Series. American Chemical Society: Washington, D.C. (1975).
Roche, E. B. Design of Biopharmaceutical Properties through Prodrugs and Analogs. American Pharmaceutical Association: Washington, D.C. (1977).
Sinkula, A. A.; Yalkowsky, S. H. J Pharm Sci. 1975, 64, 181-210.
Stella, V. J.; Charman, W. N. Naringrekar, V. H. Drugs 1985, 29, 455-473.
Bundgaard, H., ed. Design of Prodrugs. Elsevier: New York (1985).
Stella, V. J.; Himmelstein, K. J. J. Med. Chem. 1980, 23, 1275-1282.
Han, H-K; Amidon, G. L. AAPS Pharmsci 2000, 2, 1-11.
Denny, W. A. Eur. J. Med. Chem. 2001, 36, 577-595.
Wermuth, C. G. in Wermuth, C. G. ed. The Practice of Medicinal Chemistry Academic Press: San Diego (1996), 697-715.
Balant, L. P.; Doelker, E. in Wolff, M. B. ed. Burgers Medicinal Chemistry And Drug Discovery John Wiley & Sons: New York (1997), 949-982.

The metabolites of the compounds of this invention include oxidized derivatives of the compounds of Formula I, II, X, Y, Za, Zb, Zc and Zd, wherein one or more of the nitrogens are substituted with a hydroxy group; which includes derivatives where the nitrogen atom of the pyridine group is in the oxide form, referred to in the art as 1-oxo-pyridine or has a hydroxy substituent, referred to in the art as 1-hydroxy-pyridine.

General Preparative Methods

The particular process to be utilized in the preparation of the compounds used in this embodiment of the invention depends upon the specific compound desired. Such factors as the selection of the specific substituents play a role in the path to be followed in the preparation of the specific compounds of this invention. Those factors are readily recognized by one of ordinary skill in the art.

The compounds of the invention may be prepared by use of known chemical reactions and procedures as described in the following published international applications WO 00/42012, WO03/047579, WO 2005/009961, WO 2004/078747 and WO05/000284 and European patent applications EP 04023131.8 and EP 04023130.0.

The compounds of the invention can be made according to conventional chemical methods, and/or as disclosed below, from starting materials which are either commercially available or producible according to routine, conventional chemical methods. General methods for the preparation of the compounds are given below, and the preparation of representative compounds is specifically illustrated in examples.

The preparation of ureas of formula (I) can be prepared from the condensation of the two arylamine fragments and in the presence of phosgene, di-phosgene, tri-phosgene, carbonyldiimidazole, or equivalents in a solvent that does not react with any of the starting materials, as described in one or more of these published. Alternatively, compounds of formula (I) can be synthesized by reacting amino compounds) with isocyanate compounds as described in one or more of the published international applications described above.

The isocyanates are commercially available or can be synthesized from heterocyclic amines according to methods commonly known to those skilled in the art [e.g. from treatment of an amine with phosgene or a phosgene equivalent such as trichloromethyl chloroformate (diphosgene), bis(trichloromethyl)carbonate (triphosgene), or N,N′-carbonyldiimidazole (CDI); or, alternatively by a Curtius-type rearrangement of an amide, or a carboxylic acid derivative, such as an ester, an acid halide or an anhydride].

Aryl amines of formulas are commercially available, or can be synthesized according to methods commonly known to those skilled in the art. Aryl amines are commonly synthesized by reduction of nitroaryls using a metal catalyst, such as Ni, Pd, or Pt, and H₂ or a hydride transfer agent, such as formate, cyclohexadiene, or a borohydride (Rylander. Hydrogenation Methods; Academic Press: London, UK (1985)). Nitroaryls may also be directly reduced using a strong hydride source, such as LiAlH₄ (Seyden-Penne. Reductions by the Alumino- and borohydrides in Organic Synthesis; VCH Publishers: New York (1991)), or using a zero valent metal, such as Fe, Sn or Ca, often in acidic media. Many methods exist for the synthesis of nitroaryls (March. Advanced Organic Chemistry, 3^{rd} Ed.; John Wiley: New York (1985). Larock. Comprehensive Organic Transformations; VCH Publishers: New York (1989)). Nitro aryls are commonly formed by electrophilic aromatic nitration using HNO₃, or an alternative NO₂⁺ source.

Pyridine-1-oxides of Formula (I) where the pyridine ring carries a hydroxy substituent on its nitrogen atom, and A, B, L are broadly defined as above can be prepared from the corresponding pyridines using oxidation conditions know in the art. Some examples are as follows:
peracids such as meta chloroperbenzoic acids in chlorinated solvents such as dichloromethane, dichloroethane, or chloroform (Markgraf et al., Tetrahedron 1991, 47, 183);
(Me₃SiO)₂ in the presence of a catalytic amount of perrhenic acid in chlorinated solvents such as dichloromethane (Coperet et al., Terahedron Lett. 1998, 39, 761);
Perfluoro-cis-2-butyl-3-propyloxaziridine in several combinations of halogenated solvents (Amone et al., Tetrahedron 1998, 54, 7831);
Hypofluoric acid-acetonitrile complex in chloroform (Dayan et al., Synthesis 1999, 1427);
Oxone, in the presence of a base such as KOH, in water (Robker et al., J. Chem. Res., Synop. 1993, 10, 412);
Magnesium monoperoxyphthalate, in the presence of glacial acetic acid (Klemm et al., J. Heterocylic Chem. 1990, 6, 1537);
Hydrogen peroxide, in the presence of water and acetic acid (Lin A. J., Org. Prep. Proced. Int. 1991, 23(1), 114);
Dimethyldioxirane in acetone (Boyd et al., J. Chem. Soc., Perkin Trans. 1991, 9, 2189).

In addition, specific methods for preparing diaryl ureas and intermediate compounds are already described elsewhere in the patent literature, and can be adapted to the compounds of the present invention. For example, Miller S. et al, “Inhibition of p38 Kinase using Symmetrical and Unsymmetrical Diphenyl Ureas” PCT Int. Appl. WO 99 32463, Miller, S et al. “Inhibition of raf Kinase using Symmetrical and Unsymmetrical Substituted Diphenyl Ureas” PCT Int. Appl., WO 99 32436, Dumas, J. et al., “Inhibition of p38 Kinase Activity using Substituted Heterocyclic Ureas” PCT Int. Appl., WO 99 32111, Dumas, J. et al., “Method for the Treatment of Neoplasm by Inhibition of raf Kinase using N-Heteroaryl-N′-(hetero)arylureas” PCT Int. Appl., WO 99 32106, Dumas, J. et al., “Inhibition of p38 Kinase Activity using Aryl- and Heteroaryl-Substituted Heterocyclic Ureas” PCT Int. Appl., WO 99 32110, Dumas, J., et al., “Inhibition of raf Kinase using Aryl- and Heteroaryl-Substituted Heterocyclic Ureas” PCT Int. Appl., WO 99 32455, Riedl, B., et al., “O-Carboxy Aryl Substituted Diphenyl Ureas as raf Kinase Inhibitors” PCT Int. Appl., WO 00 42012, Riedl, B., et al., “O-Carboxy Aryl Substituted Diphenyl Ureas as p38 Kinase Inhibitors” PCT Int. Appl., WO 00 41698, Dumas, J. et al. “Heteroaryl ureas containing nitrogen hetero-atoms as p38 kinase inhibitors” U.S. Pat. Appl. Publ., US 20020065296, Dumas, J. et al. “Preparation of N-aryl-N′-[(acylphenoxy)phenyl]ureas as raf kinase inhibitors” PCT Int. Appl., WO 02 62763, Dumas, J. et al. “Inhibition of raf kinase using quinolyl, isoquinolyl or pyridyl ureas” PCT Int. Appl., WO 02 85857, Dumas, J. et al. “Preparation of quinolyl, isoquinolyl or pyridyl-ureas as inhibitors of raf kinase for the treatment of tumors and/or cancerous cell growth” U.S. Pat. Appl. Publ., US 20020165394. All the preceding patent applications are hereby incorporated by reference.

Synthetic transformations that may be employed in the synthesis of compounds of Formula (I) and in the synthesis of intermediates involved in the synthesis of compounds of Formula (I) are known by or accessible to one skilled in the art. Collections of synthetic transformations may be found in compilations, such as:
J. March. Advanced Organic Chemistry, 4^{th} ed.; John Wiley: New York (1992);
R. C. Larock. Comprehensive Organic Transformations, 2^{nd} ed.; Wiley-VCH: New York (1999);
F. A. Carey; R. J. Sundberg. Advanced Organic Chemistry, 2^{nd} ed.; Plenum Press: New York (1984);
T. W. Greene; P. G. M. Wuts. Protective Groups in Organic Synthesis, 3^{rd} ed.; John Wiley: New York (1999);
L. S. Hegedus. Transition Metals in the Synthesis of Complex Organic Molecules, 2^{nd} ed.; University Science Books: Mill Valley, Calif. (1994);
L. A. Paquette, Ed. The Encyclopedia of Reagents for Organic Synthesis; John Wiley: New York (1994);
A. R. Katritzky; O. Meth-Cohn; C. W. Rees, Eds. Comprehensive Organic Functional Group Transformations; Pergamon Press: Oxford, UK (1995);
G. Wilkinson; F. G A. Stone; E. W. Abel, Eds. Comprehensive Organometallic Chemistry; Pergamon Press: Oxford, UK (1982);
B. M. Trost; I. Fleming. Comprehensive Organic Synthesis; Pergamon Press: Oxford, UK (1991);
A. R. Katritzky; C. W. Rees Eds. Comprehensive Heterocylic Chemistry; Pergamon Press: Oxford, UK (1984);
A. R. Katritzky; C. W. Rees; E. F. V. Scriven, Eds. Comprehensive Heterocylic Chemistry II; Pergamon Press: Oxford, UK (1996); and
C. Hansch; P. G. Sammes; J. B. Taylor, Eds. Comprehensive Medicinal Chemistry: Pergamon Press: Oxford, UK (1990).

In addition, recurring reviews of synthetic methodology and related topics include Organic Reactions; John Wiley: New York; Organic Syntheses; John Wiley: New York; Reagents for Organic Synthesis: John Wiley: New York; The Total Synthesis of Natural Products; John Wiley: New York; The Organic Chemistry of Drug Synthesis; John Wiley: New York; Annual Reports in Organic Synthesis; Academic Press: San Diego Calif.; and Methoden der Organischen Chemie (Houben-Weyl); Thieme: Stuttgart, Germany. Furthermore, databases of synthetic transformations include Chemical Abstracts, which may be searched using either CAS OnLine or SciFinder, Handbuch der Organischen Chemie (Beilstein), which may be searched using SpotFire, and REACCS.

The compounds of Formula I have been previously characterized as having various activities, including for inhibiting the Raf/MEK/ERK pathway, c-raf, b-raf, p38, VEGFR, VEGFR2, VEGR3, FLT3, PDGFR, PDGFR-beta, and c-kit. These activities and their use in treating various diseases and conditions are disclosed in, e.g., WO 00/42021, WO 00/41698, WO03/068228, WO 03/047579, WO 2005/00996, WO 2005/000284 and U.S. Application No. 20050038080, which are hereby incorporated by reference in their entirety.

Pharmacuetical compositions intended for oral use may be prepared according to any suitable method known to the art for the manufacture of pharmaceutical compositions. The pharmaceutical composition comprises suitable administration forms which deliver the compound of the invention in a rapid manner, for example tablets (uncoated or coated tablets), tablets which disintegrate rapidly in the oral cavity or capsules optionally filled with granules (for example hard or soft gelatin capsules), sugar-coated tablets, powders, sachets, granules, pellets, dragées, chewable tablets, dispersible tables, troches and lozenges. Such compositions may contain one or more agents selected from the group consisting of diluents, sweetening agents, flavoring agents, coloring agents and preserving agents in order to provide palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; and binding agents, for example magnesium stearate, stearic acid or talc. The tablets may be uncoated or they may be coated by known techniques to delay disintegration and adsorption in the gastrointestinal tract and thereby provide a sustained action over a longer period. For example, a time delay material such as glyceryl monostearate or glyceryl distearate may be employed. These compounds may also be prepared in solid, rapidly released form.

Pharmacuetical compositions for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions containing at least one of the active materials in admixture with excipients suitable for the manufacture of aqueous suspensions may also be used. Such excipients are suspending agents, for example sodium carboxymethylcellulose, methylcellulose, hydroxypropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethylene oxycetanol, or condensation products of ethylene oxide with partial esters derived from fatty acids and hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

A pharmaceutically acceptable carrier is any carrier which is relatively non-toxic and innocuous to a patient at concentrations consistent with effective activity of the active ingredient so that any side effects ascribable to the carrier do not vitiate the beneficial effects of the active ingredient.

A pharmaceutically effective amount of compound is that amount which produces a result or exerts an influence on the particular condition being treated. The compounds of the present invention can be administered with pharmaceutically-acceptable carriers well known in the art using any effective conventional dosage unit forms, including immediate, slow and timed release preparations.

A pharmaceutically acceptable excipient is any excipient which is relatively non-toxic and innocuous to a patient at concentrations consistent with effective activity of the active ingredient so that any side effects ascribable to the excipient do not vitiate the beneficial effects of the active ingredient.

Pharmaceutically acceptable excipients according to the invention are for example disintegrants, binders, lubricants, fillers, plasticizers, surfactants and wetting agents, film-forming agents and coating materials, and coloring agents for example pigments.

Disintegrants include, but are not limited to croscarmellose sodium, crospovidone, alginic acid, carboxymethylcellulose calcium, carboxymethylcellulose sodium, microcrystalline cellulose, hydroxypropyl cellulose, low substituted hydroxypropyl cellulose, polacrillin potassium, cross-linked polyvinylpyrrolidone, sodium alginate, sodium starch glycollate, partially hydrolysed starch, sodium carboxymethyl starch and starch. Preference is given to croscarmellose sodium and/or cross-linked polyvinylpyrrolidone, more preference is given to croscarmellose sodium.

The amount of the disintegrant contained in the pharmaceutical composition of can be from 0 to 15%, preferably from 5 to 12% by the total weight of the composition.

Binders include, but are not limited to hydroxypropyl cellulose, hypromellose (hydroxypropyl methylcellulose, HPMC), microcrystalline cellulose, acacia, alginic acid, carboxymethylcellulose, ethylcellulose, methylcellulose, hydroxaethylcellulose, ethylhydroxyethylcellulose, polyvinyl alcohol, polyacrylates, carboxymethylcellulose calcium, carboxymethylcellulose sodium, compressible sugar, ethylcellulose, gelatin, liquid glucose, methylcellulose, polyvinyl pyrrolidone and pregelatinized starch. Preference is given to a hydrophilic binder which are soluble in the granulation liquid, more preference is given to hypromellose (hydroxypropyl methylcellulose, HPMC) and/or polyvinylpyrrolidone, most preference is given to hypromellose.

The amount of the binder contained in the pharmaceutical composition of can be from 0 to 15%, preferably from 0.5 to 8% by the total weight of the composition.

Lubricants include, but are not limited to calcium stearate, magnesium stearate, mineral oil, stearic acid, fumaric acid, sodium stearylfumarate, zinc stearate and polyethyleneglycol. Preference is given to magnesium stearate.

The amount of the lubricant contained in the pharmaceutical composition of can be from 0 to 2%, preferably from 0.2 to 0.8% by the total weight of the composition.

Fillers include, but are not limited to dibasic calcium phosphate, kaolin, lactose, mannitol, microcrystalline cellulose, silicated microcrystalline cellulose, dicalcium phosphate, tricalcium phosphate, magnesium trisilicate, mannitol, maltitol, sorbitol, xylitol, lactose for example the anhydrous form or the hydrate form such as the monohydrate form, dextrose, maltose, saccharose, glucose, fructose or maltodextrine, powdered cellulose, precipitated calcium carbonate, sodium carbonate, sodium phosphate and starch. Preference is given to microcrystalline cellulose, mannitol, lactose and/or dicalcium phosphate, more preference is given to microcrystalline cellulose.

The amount of the filler contained in the pharmaceutical composition of can be from 0 to 60%, preferably from 3 to 20% by the total weight of the composition.

Surfactants and Wetting agents include, but are not limited to heptadecaethylene oxycetanol, lecithins, sorbitol monooleate, polyoxyethylene sorbitol monooleate, polyoxyethylene stearate, polyoxyethylen sorbitan monolaurate, benzalkonium chloride, nonoxynol 10, oxtoxynol 9, polysorbates for example 20, 40, 60 or 80, sorbitan mono-palmitate, sodium salts of fatty alcoholsulaftes such as sodium lauryl sulfate, sodium dodecylsulfate, sodium salts of sulfosuccinates such as sodium dioctylsulfosuccinate, partially esters of fatty acids with alcohols such as glycerine monostearate, partially esters of fatty acids with sorbitans such as sorbitan monolaurate, partially esters of fatty acids with polyhydroxyethylene sorbitans such as polyethyleneglycol sorbitan monolaurate, -monostearate or -monooleate, ethers of fatty alcohols with polyhydroxyethylene, esters of fatty acids with polyhydroxyethylene, copolymers of ethylenoxide and propylenoxide (Pluronic®) and ethoxylated triglycerides. Preference is given to sodium lauryl sulfate.

The amount of the surfactant contained in the pharmaceutical composition of can be from 0 to 5%, preferably from 0.1 to 2% by the total weight of the composition.

Film-forming agents and coating materials include, but are not limited to liquid glucose, hydroxyethyl cellulose, hydroxypropyl cellulose, hydroxypropyl methylcellulose (hypromellose, HPMC), methylcellulose, ethylcellulose, cellulose acetate phthalate, shellac, polyvinylpyrrolidone, copolymers of vinylpyrrolidone and vinylacetate such as Kollidon® VA64 BASF, copolymers of acrylic- and/or methacrylic acid esters with trimethylammoniummethylacrylate, copolymers of dimethylaminomethacrylic acid and neutral methacrylic acid esters, polymers of methacrylic acid or methacrylic acid esters, copolymers of acrylic acid ethylester and methacrylic acid methyl ester, and copolymers of acrylic acid and acrylic acid methylester. Preference is given to hydroxypropyl methylcellulose (hypromellose, HPMC) as film-forming agent.

Plasticizers include, but are not limited to polyethylene glycol, diethyl phthalate and glycerol. Preference is given to polyethylene glycol.

Coloring agents include, but are not limited to pigments, inorganic pigments, FD&C Red No. 3, FD&C Red No. 20, FD&C Yellow No. 6, FD&C Blue No. 2, D&C Green No. 5, D&C Orange No. 5, D&C Red No. 8, caramel, ferric oxide red, ferric oxide yellow and titanium dioxide. Preference is given to ferric oxide red, ferric oxide yellow and titanium dioxide.

Further commonly used pharmaceutical exipients which can be used as appropriate to formulate the composition for its intended route of administration include, but is not limited to: Acidifying agents for example acetic acid, citric acid, fumaric acid, hydrochloric acid and nitric acid; alkalizing agents for example ammonia solution, ammonium carbonate, diethanolamine, monoethanolamine, potassium hydroxide, sodium borate, sodium carbonate, sodium hydroxide, triethanolamine and trolamine; adsorbents for example powdered cellulose and activated charcoal; stabilizers and antioxidants for example ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate and sodium metabisulfite; other binding materials for example block polymers, natural and synthetic rubber, polyacrylates, polyurethanes, silicones, polysiloxanes and styrene-butadiene copolymers; buffering agents for examples potassium metaphosphate, dipotassium phosphate, sodium acetate, sodium citrate anhydrous and sodium citrate hydrates; encapsulating agents for example gelatin, starch and cellulose derivates); flavorants, masking agents and odors for example anise oil, cinnamon oil, cocoa, menthol, orange oil, peppermint oil and vanillin; humectants for example glycerol, propylene glycol and sorbitol; sweeteners for example aspartame, dextrose, glycerol, mannitol, propylene glycol, saccharin sodium, sorbitol and sucrose; anti-adherents for example magnesium stearate and talc; direct compression excipients for example dibasic calcium phosphate, lactose and microcrystalline cellulose; tablet polishing agents for example carnauba wax and white wax.

Commonly used pharmaceutical ingredients which can be used as appropriate to formulate the composition for its intended route of administration include:

acidifying agents (examples include but are not limited to acetic acid, citric acid, fumaric acid, hydrochloric acid, nitric acid);

alkalinizing agents (examples include but are not limited to ammonia solution, ammonium carbonate, diethanolamine, monoethanolamine, potassium hydroxide, sodium borate, sodium carbonate, sodium hydroxide, triethanolamine, trolamine);

adsorbents (examples include but are not limited to powdered cellulose and activated charcoal);

aerosol propellants (examples include but are not limited to carbon dioxide, CCl₂F₂, F₂ClC-CClF₂ and CClF₃)

air displacement agents (examples include but are not limited to nitrogen and argon);

antifungal preservatives (examples include but are not limited to benzoic acid, butylparaben, ethylparaben, methylparaben, propylparaben, sodium benzoate);

antimicrobial preservatives (examples include but are not limited to benzalkonium chloride, benzethonium chloride, benzyl alcohol cetylpyridinium chloride, chlorobutanol, phenol, phenylethyl alcohol, phenylmercuric nitrate and thimerosal);

antioxidants (examples include but are not limited to ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite);

binding materials (examples include but are not limited to block polymers, natural and synthetic rubber, polyacrylates, polyurethanes, silicones, polysiloxanes and styrene-butadiene copolymers);

buffering agents (examples include but are not limited to potassium metaphosphate, dipotassium phosphate, sodium acetate, sodium citrate anhydrous and sodium citrate dihydrate)

carrying agents (examples include but are not limited to acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection)

chelating agents (examples include but are not limited to edetate disodium and edetic acid)

colorants (examples include but are not limited to FD&C Red No. 3, FD&C Red No. 20, FD&C Yellow No. 6, FD&C Blue No. 2, D&C Green No. 5, D&C Orange No. 5, D&C Red No. 8, caramel and ferric oxide red);

clarifying agents (examples include but are not limited to bentonite);

emulsifying agents (examples include but are not limited to acacia, cetomacrogol, cetyl alcohol, glyceryl monostearate, lecithin, sorbitan monooleate, polyoxyethylene 50 monostearate);

encapsulating agents (examples include but are not limited to gelatin and cellulose acetate phthalate)

flavorants (examples include but are not limited to anise oil, cinnamon oil, cocoa, menthol, orange oil, peppermint oil and vanillin);

humectants (examples include but are not limited to glycerol, propylene glycol and sorbitol);

levigating agents (examples include but are not limited to mineral oil and glycerin);

oils (examples include but are not limited to arachis oil, mineral oil, olive oil, peanut oil, sesame oil and vegetable oil);

ointment bases (examples include but are not limited to lanolin, hydrophilic ointment, polyethylene glycol ointment, petrolatum, hydrophilic petrolatum, white ointment, yellow ointment, and rose water ointment);

penetration enhancers (transdermal delivery) (examples include but are not limited to monohydroxy or polyhydroxy alcohols, mono- or polyvalent alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas)

plasticizers (examples include but are not limited to diethyl phthalate and glycerol);

solvents (examples include but are not limited to ethanol, corn oil, cottonseed oil, glycerol, isopropanol, mineral oil, oleic acid, peanut oil, purified water, water for injection, sterile water for injection and sterile water for irrigation);

stiffening agents (examples include but are not limited to cetyl alcohol, cetyl esters wax, microcrystalline wax, paraffin, stearyl alcohol, white wax and yellow wax);

suppository bases (examples include but are not limited to cocoa butter and polyethylene glycols (mixtures));

surfactants (examples include but are not limited to benzalkonium chloride, nonoxynol 10, oxtoxynol 9, polysorbate 80, sodium lauryl sulfate and sorbitan mono-palmitate);

suspending agents (examples include but are not limited to agar, bentonite, carbomers, carboxymethylcellulose sodium, hydroxyethyl cellulose, hydroxypropyl cellulose, hydroxypropyl methylcellulose, kaolin, methylcellulose, tragacanth and veegum);

sweetening agents (examples include but are not limited to aspartame, dextrose, glycerol, mannitol, propylene glycol, saccharin sodium, sorbitol and sucrose);

tablet anti-adherents (examples include but are not limited to magnesium stearate and talc);

tablet binders (examples include but are not limited to acacia, alginic acid, carboxymethylcellulose sodium, compressible sugar, ethylcellulose, gelatin, liquid glucose, methylcellulose, non-crosslinked polyvinyl pyrrolidone, and pregelatinized starch);

tablet and capsule diluents (examples include but are not limited to dibasic calcium phosphate, kaolin, lactose, mannitol, microcrystalline cellulose, powdered cellulose, precipitated calcium carbonate, sodium carbonate, sodium phosphate, sorbitol and starch);

tablet coating agents (examples include but are not limited to liquid glucose, hydroxyethyl cellulose, hydroxypropyl cellulose, hydroxypropyl methylcellulose, methylcellulose, ethylcellulose, cellulose acetate phthalate and shellac);

tablet direct compression excipients (examples include but are not limited to dibasic calcium phosphate);

tablet disintegrants (examples include but are not limited to alginic acid, carboxymethylcellulose calcium, microcrystalline cellulose, polacrillin potassium, cross-linked polyvinylpyrrolidone, sodium alginate, sodium starch glycollate and starch);

tablet glidants (examples include but are not limited to colloidal silica, corn starch and talc);

tablet lubricants (examples include but are not limited to calcium stearate, magnesium stearate, mineral oil, stearic acid and zinc stearate);

tablet/capsule opaquants (examples include but are not limited to titanium dioxide);

tablet polishing agents (examples include but are not limited to carnauba wax and white wax);

thickening agents (examples include but are not limited to beeswax, cetyl alcohol and paraffin);

tonicity agents (examples include but are not limited to dextrose and sodium chloride);

viscosity increasing agents (examples include but are not limited to alginic acid, bentonite, carbomers, carboxymethylcellulose sodium, methylcellulose, polyvinyl pyrrolidone, sodium alginate and tragacanth); and

wetting agents (examples include but are not limited to heptadecaethylene oxycetanol, lecithins, sorbitol monooleate, polyoxyethylene sorbitol monooleate, and polyoxyethylene stearate).

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example, sweetening, flavoring and coloring agents, may also be present.

The pharmacuetical compositions may also be in the form of non-aqueous liquid formulations, e.g., oily suspensions which may be formulated by suspending the active ingredients in polyethyleneglycol, a vegetable oil, for example arachis oil, olive oil, sesame oil or peanut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These compositions may be preserved by the addition of an anti-oxidant such as ascorbic acid.

Pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The active ingredients of the drug combination of the invention may also be administered transdermally using methods known to those skilled in the art (see, for example: Chien; “Transdermal Controlled Systemic Medications”; Marcel Dekker, Inc.; 1987. Lipp et al. WO94/04157 3 Mar. 1994). For example, a solution or suspension of an aryl urea compound in a suitable volatile solvent optionally containing penetration enhancing agents can be combined with additional additives known to those skilled in the art, such as matrix materials and bacteriocides. After sterilization, the resulting mixture can be formulated following known procedures into dosage forms. In addition, on treatment with emulsifying agents and water, a solution or suspension of an aryl urea compound may be formulated into a lotion or salve.

Suitable solvents for processing transdermal delivery systems are known to those skilled in the art, and include dimethylsulfoxide, lower alcohols such as ethanol or isopropyl alcohol, lower ketones such as acetone, lower carboxylic acid esters such as ethyl acetate, polar ethers such as tetrahydrofuran, lower hydrocarbons such as hexane, cyclohexane or benzene, or halogenated hydrocarbons such as dichloromethane, chloroform, trichlorotrifluoroethane, or trichlorofluoroethane. Suitable solvents may also include mixtures of one or more materials selected from lower alcohols, lower ketones, lower carboxylic acid esters, polar ethers, lower hydrocarbons, halogenated hydrocarbons.

Suitable penetration enhancing materials for transdermal delivery systems are known to those skilled in the art, and include, for example, monohydroxy or polyhydroxy alcohols such as ethanol, propylene glycol or benzyl alcohol, saturated or unsaturated C₈-C₁₈ fatty alcohols such as lauryl alcohol or cetyl alcohol, saturated or unsaturated C₈-C₁₈ fatty acids such as stearic acid, saturated or unsaturated fatty esters with up to 24 carbons such as methyl, ethyl, propyl, isopropyl, n-butyl, sec-butyl, isobutyl, tertbutyl or monoglycerin esters of acetic acid, capronic acid, lauric acid, myristinic acid, stearic acid, or palmitic acid, or diesters of saturated or unsaturated dicarboxylic acids with a total of up to 24 carbons such as diisopropyl adipate, diisobutyl adipate, diisopropyl sebacate, diisopropyl maleate, or diisopropyl fumarate. Additional penetration enhancing materials include phosphatidyl derivatives such as lecithin or cephalin, terpenes, amides, ketones, ureas and their derivatives, and ethers such as dimethyl isosorbid and diethyleneglycol monoethyl ether. Suitable penetration enhancing formulations may also include mixtures of one or more materials selected from monohydroxy or polyhydroxy alcohols, saturated or unsaturated C₈-C₁₈ fatty alcohols, saturated or unsaturated C₈-C₁₈ fatty acids, saturated or unsaturated fatty esters with up to 24 carbons, diesters of saturated or unsaturated discarboxylic acids with a total of up to 24 carbons, phosphatidyl derivatives, terpenes, amides, ketones, ureas and their derivatives, and ethers.

Suitable binding materials for transdermal delivery systems are known to those skilled in the art and include polyacrylates, silicones, polyurethanes, block polymers, styrenebutadiene copolymers, and natural and synthetic rubbers. Cellulose ethers, derivatized polyethylenes, and silicates may also be used as matrix components. Additional additives, such as viscous resins or oils may be added to increase the viscosity of the matrix.

Specific preparations of compounds within the drug combination of this invention can be adapted from others known in the art. For example, Riedl, B., et al., “O-Carboxy Aryl Substituted Diphenyl Ureas as raf Kinase Inhibitors” PCT Int. Appl., WO 00 42012, Riedl, B., et al., “O-Carboxy Aryl Substituted Diphenyl Ureas as p38 Kinase Inhibitors” PCT Int. Appl., WO 00 41698, incorporated herein by reference.

Pharmaceutical compositions according to the present invention can be illustrated as follows:

Sterile IV Solution: A 5 mg/ml solution of a desired compound of the drug combination of this invention is made using sterile, injectable water, and the pH is adjusted if necessary. The solution is diluted for administration to 1-2 mg/ml with sterile 5% dextrose and is administered as an IV infusion over 60 minutes.

Lyophilized powder for IV administration: A sterile preparation can be prepared with (i) 100-1000 mg of a desired compound of the drug combination of this invention as a lyophilized powder, (ii) 32-327 mg/ml sodium citrate, and (iii) 300-3000 mg Dextran 40. The formulation is reconstituted with sterile, injectable saline or dextrose 5% to a concentration of 10 to 20 mg/ml, which is further diluted with saline or dextrose 5% to 0.2-0.4 mg/ml, and is administered either IV bolus or by IV infusion over 15-60 minutes.

Intramuscular suspension: The following solution or suspension can be prepared, for intramuscular injection:

50 mg/ml of the desired, water-insoluble compound of the drug combination of this invention
5 mg/ml sodium carboxymethylcellulose
4 mg/ml TWEEN 80
9 mg/ml sodium chloride
9 mg/ml benzyl alcohol
   Hard Shell Capsules: A large number of unit capsules are prepared by filling standard two-piece hard galantine capsules each with 100 mg of powdered active ingredient, 150 mg of lactose, 50 mg of cellulose and 6 mg of magnesium stearate.
   Soft Gelatin Capsules: A mixture of active ingredient in a digestible oil such as soybean oil, cottonseed oil or olive oil is prepared and injected by means of a positive displacement pump into molten gelatin to form soft gelatin capsules containing 100 mg of the active ingredient. The capsules are washed and dried. The active ingredient can be dissolved in a mixture of polyethylene glycol, glycerin and sorbitol to prepare a water miscible medicine mix.
   Tablets: A large number of tablets are prepared by conventional procedures so that the dosage unit was 100 mg of active ingredient, 0.2 mg. of colloidal silicon dioxide, 5 mg of magnesium stearate, 275 mg of microcrystalline cellulose, 11 mg. of starch, and 98.8 mg of lactose. Appropriate aqueous and non-aqueous coatings may be applied to increase palatability, improve elegance and stability or delay absorption.

Immediate Release Tablets/Capsules: These are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid-state extraction techniques. The drug compounds may be compressed with viscoelastic and thermoelastic sugars and polymers or effervescent components to produce porous matrices intended for immediate release, without the need of water.

The invention also encompasses kits for treating mammalian cancers. Such kits can be used to treat a patient with a raf kinase stimulated cancer as well as cancers not stimulated through raf kinase. The kit can comprise a single pharmaceutical formulation containing a substititued diaryl urea compound of Formula I (e.g., Sorafenib) and taxane, (e.g., paclitaxel) and a platinum complex (e.g., carboplatin). Alternatively, the kit can comprise a substituted diaryl urea compound a taxane and platinum complex in separate formulations. The kit can also include instructions for how to administer the compounds to a patient with cancer in need of treatment. The kit can be used to treat different cancer types which include but are not limited to NSCLC, colon, prostate, leukemia, melanoma, hepatocellular, renal, head and neck, glioma, lung, pancreatic, ovarian, and mammary.

It will be appreciated by those skilled in the art that the particular method of administration will depend on a variety of factors, all of which are routinely considered when administering therapeutics. It will also be understood, however, that the specific dose level for any given patient will depend upon a variety of factors, including, the activity of the specific compound employed, the age of the patient, the body weight of the patient, the general health of the patient, the gender of the patient, the diet of the patient, time of administration, route of administration, rate of excretion, drug combinations, and the severity of the condition undergoing therapy. It will be further appreciated by one skilled in the art that the optimal course of treatment, i.e., the mode of treatment and the daily number of doses of one or more of the drugs within the combination (or a pharmaceutically acceptable salt thereof) given for a defined number of days, can be ascertained by those skilled in the art using conventional treatment tests.

Based upon standard laboratory techniques known to evaluate compounds useful for the treatment of hyper-proliferative disorders, by standard toxicity tests and by standard pharmacological assays for the determination of treatment of the conditions identified above in mammals, and by comparison of these results with the results of known medicaments that are used to treat these conditions, the effective dosage of the compounds of this invention can readily be determined for treatment of each desired indication. The amount of the active ingredient to be administered in the treatment of one of these conditions can vary widely according to such considerations as the particular compound and dosage unit employed, the mode of administration, the period of treatment, the age and gender of the patient treated, and the nature and extent of the condition treated.

Generally, the use of the drug combination of this invention will serve to (1) yield better efficacy in reducing the growth of a tumor or even eliminate the tumor as compared to administration of a single chemotherapeutic agent, (2) provide for the administration of lesser amounts of the administered chemotherapeutic agents, (3) provide for a chemotherapeutic treatment that is well tolerated in the patient with less deleterious pharmacological complications resulting from larger doses of single chemotherapies and certain other combined therapies, (4) provide for treating a broader spectrum of different cancer types in mammals, especially humans, (5) provide for a higher response rate among treated patients, (6) provide for a longer survival time among treated patients compared to standard chemotherapy treatments, (7) provide a longer time for tumor progression, and/or (8) yield efficacy and tolerability results at least as good as those of the agents used alone, compared to known instances where other cancer agent combinations produce antagonist effects.

The substituted diaryl urea compound of Formula I can be administered to a patient at a dosage which can range from about 0.001 to about 300 mg/Kg of total body weight. A unit dosage may contain from about 0.5 mg to about 2000 mg of active ingredient, and can be administered one or more times per day. Preference is given to an amount of the substituted diarylurea compound of formula (I) in the pharmaceutical composition from 20 to 2000 mg, preferably from 40 to 800 mg, more preferably from 50 to 600 mg. Particular preference is given to an amount of p-toluenesulfonic acid salt of 4{4-[3-(4-chloro-3-trifluoromethylphenyl)-ureido]-phenoxy}-pyridine-2-carboxylic acid methyl amide in the pharmaceutical composition from 27 to 2740 mg, preferably from 54 to 1096, more preferably from 68 to 822 mg.

The daily dose for oral administration will preferably be from 0.1 to 300 mg/kg of total body weight, typically from about 0.1 mg/kg to about 50 mg/kg body weight per day. The daily dosage for administration by injection which includes intravenous, intramuscular, subcutaneous and parenteral injection as well as infusion techniques will preferably be from 0.1 to 300 mg/kg of total body weight. The daily vaginal dosage regime will preferably be from 0.1 to 300 mg/kg of total body weight. The daily topical dosage regimen will preferably be from 0.1 to 300 mg administered between one to four times daily. The transdermal concentration will preferably be that required to maintain a daily dose of from 1 to 300 mg/kg. For all the above mentioned routes of administration, the preferred dosage is 0.1 to 300 mg/kg. The daily inhalation dosage regimen will preferably be from 0.1 to 300 mg/kg of total body weight.

The taxanes such as paclitaxel and platinum complexes such as carboplatin are each preferably administered non-orally, more preferably by intravenous infusion, in conventional amounts routinely used in cancer monotherapy or reduced amounts based on the combination of active agents.

Based on body surface area, the infusion dosage of paclitaxel may range from about 10 to 300 mg/m², preferably from about 30 to 200 mg/m² and more preferably about 100, 135 or 175 mg/m². Paclitaxel infusions should be preceded with appropriate premedications known to those skilled in the art. The paclitaxel dosage is preferably administered intravenously by infusion over a period of at least about 3 hours, preferably over a period of about 3 or 24 hours. The carboplatin dosage is preferably administered intravenously by infusion, preferably over a period of at least about 15 minutes. The infusion dosage of carboplatin may range from about 100 to 500 mg/m² preferably about 300 or 360 mg/m².

The combined dosages of carboplatin and paclitaxel can range from 50 to 500 mg/m², 100 to 400 mg/m², 150 to 300 mg/m², 175-275 mg/m² and 200-250 mg/m² for a single infusion.

For each of the substituted diaryl urea compounds of Formula I, taxanes and platinum complexes, the administered dosage of the compound may be modified depending on any superior or unexpected results which may be obtained as routinely determined with this invention. Based upon standard laboratory techniques known to evaluate compounds useful for the treatment of hyper-proliferative disorders, by standard toxicity tests and by standard pharmacological assays for the determination of treatment of the conditions identified above in mammals, and by comparison of these results with the results of known medicaments that are used to treat these conditions, the effective dosage of the pharmaceutical compositions of this invention can readily be determined by those skilled in the art. The amount of the administered active ingredient can vary widely according to such considerations as the particular compound and dosage unit employed, the mode and time of administration, the period of treatment, the age, sex, and general condition of the patient treated, the nature and extent of the condition treated, the rate of drug metabolism and excretion, the potential drug combinations and drug-drug interactions, and the like.

The substituted diaryl urea compounds of Formula I can be administered orally, topically, parenterally, rectally, by inhalation, and by injection. Administration by injection includes intravenous, intramuscular, subcutaneous, and parenterally as well as by infusion techniques. The aryl urea compound can be present in association with one or more non-toxic pharmaceutically acceptable carriers and if desired other active ingredients. A preferred route of administration for the aryl urea compound is oral administration.

The taxanes and platinum complexes can be administered to a patient by any of the conventional routes of administration for these compounds. This can include oral, topical, parenteral, rectal and inhalation administration as well as injection. Administration by injection includes intravenous, intramuscular, subcutaneous, and parenterally as well as by infusion techniques. The preferred route of administration for the taxanes and platinum complexes used in this invention is typically by injection which is the same route of administration used for the agent alone. Any of the taxanes and platinum complexes can be administered in combination with a substituted diaryl urea compound by any of the mentioned routes of administration.

For administering the substituted diaryl urea compound of Formula I and both the taxane and platinum complex, by any of the routes of administration herein discussed) the substituted diaryl urea compound can be administered simultaneously with the taxane and platinum complex. This can be performed by administering a single formulation which contains both the substituted diaryl urea compound and the taxane and platinum complex or administering the substituted diaryl urea compound and the taxane and platinum complex in independent formulations at the same time to a patient.

Alternatively, the substituted diaryl urea compound of Formula I can be administered in tandem with the taxane and platinum complex. The substituted diaryl urea compound can be administered prior to either the taxane, the platinum complex or both. For example, the substituted aryl urea compound can be administered once or more times per day up to 28 consecutive days followed by administration of the taxane and platinum complex. Also, either the taxane, platinum complex or both can be administered first followed by administration of the substituted diaryl urea compound. The choice of sequence administration of the substituted diaryl urea compound relative to the taxane and platinum complex may vary for different agents. Also, the taxane and platinum complex can be administered using any regimen which is conventionally used for these agents.

In another regimen of administration, the substituted diaryl urea compound of Formula I and the taxane and platinum complex can be administered one or more times per day on the day of administration.

Method of Treating Hyper-Proliferative Disorders

The present invention also relates to a method for using the pharmaceutical composition according to the invention to treat mammalian hyper-proliferative disorders, including cancer. This method comprises administering a pharmaceutical composition containing the drug combination to a mammal in need thereof, including a human, an amount which is effective to treat the disorder.

The term “hyper-proliferative disorders” and/or “cancer” not only refers to solid tumors, such as cancers of the breast, respiratory tract, brain, reproductive organs, digestive tract, urinary tract, eye, liver, skin, head and neck, thyroid, parathyroid and their distant metastases, but also includes lymphomas, sarcomas, and leukaemias.

Drug combinations of the present invention can be utilized to treat any diseases or conditions that are associated with, or mediated by, the cellular pathways modulated by the compounds comprising the combinations. These pathways, include, but are not limited to signaling pathways which comprise, e.g., VEGFR, VEGFR2, Raf/Mek/Erk, Akt/PI3K, MTOR, PTEN, etc. (see also above). The drug combinations can be useful to treat diseases that are associated with, or mediated by, mutations in one of more genes present in these pathways, including cancer-associated mutations in PTEN, ras, Raf, Akt, PI3K, etc.

Although the compounds may be known as specific inhibitors, the present invention includes any ameliorative or therapeutic effect, regardless of the mechanism of action or how it is achieved.

The drug combination can have one or more of the following activities, including, anti-proliferative; anti-tumor; anti-angiogenic; inhibiting the proliferation of endothelial or tumor cells; anti-neoplastic; immunosuppressive; immunomodulatory; apoptosis-promoting, etc.

Conditions or diseases that can be treated in accordance with the present invention include proliferative disorders such as cancer.

Any tumor or cancer can be treated, including, but not limited to, cancers having one or more mutations in raf, VEGFR-2, VEGFR-3, PDGFR-beta, Flt-3, ras, PTEN, Akt, PI3K, mTOR, as well as any upstream or downstream member of the signaling pathways of which they are a part. A tumor or cancer can be treated with a compound of the present invention irrespective of the mechanism that is responsible for it. Cancers of any organ can be treated, including cancers of, but are not limited to, e.g., colon, pancreas, breast, prostate, bone, liver, kidney, lung, testes, skin, pancreas, stomach, prostate, ovary, uterus, head and neck, blood cell, lymph, etc.

Cancers that can be treated in accordance with the present invention include, especially, but not limited to, brain tumors, breast cancer, bone sarcoma (e.g., osteosarcoma and Ewings sarcoma), bronchial premalignancy, endometrial cancer, glioblastoma, hematologic malignancies, hepatocellular carcinoma, Hodgkin's disease, kidney neoplasms, leukemia, leimyosarcoma, liposarcoma, lymphoma, Lhermitte-Duclose disease, malignant glioma, melanoma, malignant melanoma, metastases, multiple myeloma, myeloid metaplasia, myeloplastic syndromes, non-small cell lung cancer, pancreatic cancer, prostate cancer, renal cell carcinoma (e.g., advanced, advanced refractory), rhabdomyosarcoma, soft tissue sarcoma, squamous epithelial carcinoma of the skin, cancers associated with loss of function of PTEN; activated Akt (e.g. PTEN null tumors and tumors with ras mutations).

Examples of breast cancer include, but are not limited to, invasive ductal carcinoma, invasive lobular carcinoma, ductal carcinoma in situ, and lobular carcinoma in situ.

Examples of cancers of the respiratory tract include, but are not limited to, small-cell, non-small-cell lung carcinoma, bronchial adenoma, and pleuropulmonary blastoma.

Examples of brain cancers include, but are not limited to, brain stem and hypophtalmic glioma, cerebellar and cerebral astrocytoma, medulloblastoma, ependymoma, and neuroectodermal and pineal tumor.

Tumors of the male reproductive organs include, but are not limited to, prostate and testicular cancer. Tumors of the female reproductive organs include, but are not limited to, endometrial, cervical, ovarian, vaginal, and vulvar cancer, as well as sarcoma of the uterus.

Tumors of the digestive tract include, but are not limited to, anal, colon, colorectal, esophageal, gallbladder, gastric, pancreatic, rectal, small intestine, and salivary gland cancers.

Tumors of the urinary tract include, but are not limited to, bladder, penile, kidney, renal pelvis, ureter, and urethral cancers.

Eye cancers include, but are not limited to, intraocular melanoma and retinoblastoma.

Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to, squamous cell carcinoma, Kaposi's sarcoma, malignant melanoma, Merkel cell skin cancer, and non-melanoma skin cancer.

Head-and-neck cancers include, but are not limited to, laryngeal, hypopharyngeal, nasopharyngeal, and/or oropharyngeal cancers, and lip and oral cavity cancer.

Lymphomas include, but are not limited to, AIDS-related lymphoma, non-Hodgkin's lymphoma, cutaneous T-cell lymphoma, Hodgkin's disease, and lymphoma of the central nervous system.

Sarcomas include, but are not limited to, sarcoma of the soft tissue, osteosarcoma, malignant fibrous histiocytoma, lymphosarcoma, and rhabdomyosarcoma.

Leukemias include, but are not limited to, acute myeloid leukemia, acute lymphoblastic leukemia, chronic lymphocytic leukemia, chronic myelogenous leukemia, and hairy cell leukemia.

In addition to inhibiting the proliferation of tumor cells, compounds of the present invention can also cause tumor regression, e.g., a decrease in the size of a tumor, or in the extent of cancer in the body.

In addition, the drug combinations can be administered to modulate one or more the following processes, cell growth (e.g., proliferation), tumor cell growth (including, e.g., differentiation, cell survival, and/or proliferation), tumor regression, endothelial cell growth (including, e.g., differentiation, cell survival, and/or proliferation), angiogenesis (blood vessel growth), angiogenesis, and/or hematopoiesis (e.g., proliferation, T-cell development, etc.).

(The drugs can also be administered sequentially at different times. Agents can be formulated conventionally to achieve the desired rates of release over extended period of times, e.g., 12-hours, 24-hours. This can be achieved by using agents and/or their derivatives which have suitable metabolic half-lives, and/or by using controlled release formulations.

The drug combinations can be synergistic, e.g., where the joint action of the drugs is such that the combined effect is greater than the algebraic sum of their individual effects. Thus, reduced amounts of the drugs can be administered, e.g., reducing toxicity or other deleterious or unwanted effects, and/or using the same amounts as used when the agents are administered alone, but achieving greater efficacy, e.g., in having more potent antiproliferative and pro-apoptotic action.

Compounds of the present invention can be further combined with any other suitable additive or pharmaceutically acceptable carrier. Such additives include any of the substances already mentioned, as well as any of those used conventionally, such as those described in Remington: The Science and Practice of Pharmacy (Gennaro and Gennaro, eds, 20th edition, Lippincott Williams & Wilkins, 2000); Theory and Practice of Industrial Pharmacy (Lachman et al., eds., 3rd edition, Lippincott Williams & Wilkins, 1986); Encyclopedia of Pharmaceutical Technology (Swarbrick and Boylan, eds., 2nd edition, Marcel Dekker, 2002). These can be referred to herein as “pharmaceutically acceptable carriers” to indicate they are combined with the active drug and can be administered safely to a subject for therapeutic purposes.

In addition, compounds of the present invention can be administered with other active agents or therapies (e.g., radiation) that are utilized to treat any of the above-mentioned diseases and/or conditions.

Assays

Activity of combinations of the present invention can be determined according to any effective in vitro or in vivo method.

Kinase Activity

Kinase activity can be determined routinely using conventional assay methods. Kinase assays typically comprise the kinase enzyme, substrates, buffers, and components of a detection system. A typical kinase assay involves the reaction of a protein kinase with a peptide substrate and an ATP, such as ³²P-ATP, to produce a phosphorylated end-product (for instance, a phosphoprotein when a peptide substrate is used). The resulting end-product can be detected using any suitable method. When radioactive ATP is utilized, a radioactively labeled phosphoprotein can be separated from the unreacted gamma-³²P-ATP using an affinity membrane or gel electrophoresis, and then visualized on the gel using autoradiography or detected with a scintillation counter. Non-radioactive methods can also be used. Methods can utilize an antibody which recognizes the phosphorylated substrate, e.g., an anti-phosphotyrosine antibody. For instance, kinase enzyme can be incubated with a substrate in the presence of ATP and kinase buffer under conditions which are effective for the enzyme to phosphorylate the substrate. The reaction mixture can be separated, e.g., electrophoretically, and then phosphorylation of the substrate can be measured, e.g., by Western blotting using an anti-phosphotyrosine antibody. The antibody can be labeled with a detectable label, e.g., an enzyme, such as HRP, avidin or biotin, chemiluminescent reagents, etc. Other methods can utilize ELISA formats, affinity membrane separation, fluorescence polarization assays, luminescent assays, etc.

An alternative to a radioactive format is time-resolved fluorescence resonance energy transfer (TR-FRET). This method follows the standard kinase reaction, where a substrate, e.g., biotinylated poly(GluTyr), is phosphorylated by a protein kinase in the presence of ATP. The end-product can then detected with a europium chelate phosphospecific antibody (anti-phosphotyrosine or phosphoserine/threonine), and streptavidin-APC, which binds the biotinylated substrate. These two components are brought together spatially upon binding, and energy transfer from the phosphospecific antibody to the acceptor (SA-APC) produces fluorescent readout in the homogeneous format.

Raf/MEK/ERK Activity

A c-Raf kinase assay can be performed with a c-Raf enzyme activated (phosphorylated) by Lck kinase. Lck-activated c-Raf (Lck/c-Raf) is produced in Sf9 insect cells by co-infecting cells with baculoviruses expressing, under the control of the polyhedrin promoter, GST-c-Raf (from amino acid 302 to amino acid 648) and Lck (full-length). Both baculoviruses are used at the multiplicity of infection of 2.5 and the cells are harvested 48 hours post infection.

MEK-1 protein is produced in Sf9 insect cells by infecting cells with the baculovirus expressing GST-MEK-1 (full-length) fusion protein at the multiplicity of infection of 5 and harvesting the cells 48 hours post infection. Similar purification procedure is used for GST-c-Raf 302-648 and GST-MEK-1.

Transfected cells are suspended at 100 mg of wet cell biomass per mL in a buffer containing 10 mM sodium phosphate, 140 mM sodium chloride pH 7.3, 0.5% Triton X-100 and the protease inhibitor cocktail. The cells are disrupted with a Polytron homogenizer and centrifuged 30,000 g for 30 minutes. The 30,000 g supernatant is applied applied onto GSH-Sepharose. The resin is washed with a buffer containing 50 mM Tris, pH 8.0, 150 mM NaCl and 0.01% Triton X-100. The GST-tagged proteins are eluted with a solution containing 100 mM Glutathione, 50 mM Tris, pH 8.0, 150 mM NaCl and 0.01% Triton X-100. The purified proteins are dialyzed into a buffer containing 20 mM Tris, pH 7.5, 150 mM NaCl and 20% Glycerol.

Test compounds are serially diluted in DMSO using three-fold dilutions to stock concentrations ranging typically from 50 μM to 20 nM (e.g., final concentrations in the assay can range from 1 μM to 0.4 nM). The c-Raf biochemical assay is performed as a radioactive filtermat assay in 96-well Costar polypropylene plates (Costar 3365). The plates are loaded with 75 μL solution containing 50 mM HEPES pH 7.5, 70 mM NaCl, 80 ng of Lck/c-Raf and 1 μg MEK-1. Subsequently, 2 μL of the serially diluted individual compounds is added to the reaction, prior to the addition of ATP. The reaction is initiated with 25 μL ATP solution containing 5 μM ATP and 0.3 μCi [33P]-ATP. The plates were sealed and incubated at 32° C. for 1 hour. The reaction is quenched with the addition of 50 μl of 4% Phosphoric Acid and harvested onto P30 filtermats (PerkinElmer) using a Wallac Tomtec Harvester. Filtermats are washed with 1% Phosphoric Acid first and deinonized H₂O second. The filters are dried in a microwave, soaked in scintillation fluid and read in a Wallac 1205 Betaplate Counter (Wallac Inc., Atlanta, Ga., U.S.A.). The results are expressed as percent inhibition.
% Inhibition=[100−(Tib/Ti)]×100 where
Tib=(counts per minute with inhibitor)−(background)
Ti=(counts per minute without inhibitor)−(background)

Raf activity can also be monitored by its ability to initiate the cascade leading to ERK phosphorylation (i.e., raf/MEK/ERK), resulting in phospho-ERK. A Bio-Plex Phospho-ERK1/2 immunoassay can be performed as follows:

A 96-well phospho-ERK (pERK) immunoassay, using laser flow cytometry platform has been established to measure inhibition of basal pERK in cell lines. MDA-MB-231 cells are plated at 50,000 cells per well in 96-well microtitre plates in complete growth media. For effects of test compounds on basal pERK1/2 inhibition, the next day after plating, MDA-MB-231 cells are transferred to DMEM with 0.1% BSA and incubated with test compounds diluted 1:3 to a final concentration of 3 mM to 12 nM in 0.1% DMSO. Cells are incubated with test compounds for 2 h, washed, and lysed in Bio-Plex whole cell lysis buffer A. Samples are diluted with buffer B1:1 (v/v) and directly transferred to assay plate or frozen at −80 C degrees until processed. 50 mL of diluted MDA-MB-231 cell lysates are incubated with about 2000 of 5 micron Bio-Plex beads conjugated with an anti-ERK1/2 antibody overnight on a shaker at room temperature. The next day, biotinylated phospho-ERK1/2 sandwich immunoassay is performed, beads are washed 3 times during each incubation and then 50 mL of PE-strepavidin is used as a developing reagent. The relative fluorescence units of pERK1/2 is detected by counting 25 beads with Bio-Plex flow cell (probe) at high sensitivity. The IC50 is calculated by taking untreated cells as maximum and no cells (beads only) as background.

Cell Proliferation

An example of a cell proliferation assay is described in the Examples below. However, proliferation assays can be performed by any suitable method. For example, a breast carcinoma cell proliferation assay can be performed as follows. Other cell types can be substituted for the MDA-MB-231 cell line.

Human breast carcinoma cells (MDA MB-231, NCI) are cultured in standard growth medium (DMEM) supplemented with 10% heat-inactivated FBS at 37° C. in 5% CO₂ (vol/vol) in a humidified incubator. Cells are plated at a density of 3000 cells per well in 90 μL growth medium in a 96 well culture dish. In order to determine T₀ₕ CTG values, 24 hours after plating, 100 μL of CellTiter-Glo Luminescent Reagent (Promega) is added to each well and incubated at room temperature for 30 minutes. Luminescence is recorded on a Wallac Victor II instrument. The CellTiter-Glo reagent results in cell lysis and generation of a luminescent signal proportional to the amount of ATP present, which, in turn is directly proportional to the number of cells present.

Test compounds are dissolved in 100% DMSO to prepare 10 mM stocks. Stocks are further diluted 1:400 in growth medium to yield working stocks of 25 μM test compound in 0.25% DMSO. Test compounds are serially diluted in growth medium containing 0.25% DMSO to maintain constant DMSO concentrations for all wells. 60 μL of diluted test compound are added to each culture well to give a final volume of 180 μL. The cells with and without individual test compounds are incubated for 72 hours at which time ATP dependent luminescence was measured, as described previously, to yield T₇₂ₕ values. Optionally, the IC₅₀ values can be determined with a least squares analysis program using compound concentration versus percent inhibition.
% Inhibition=[1−(T_{72h test}−T₀ₕ)/(T_{72h ctrl}−T₀ₕ)]×100, where
T_{72h test}=ATP dependent luminescence at 72 hours in the presence of test compound
T_{72h ctrl}=ATP dependent luminescence at 72 hours in the absence of test compound
T₀ₕ=ATP dependent luminescence at Time Zero.
Angiogenesis

One useful model to study angiogenesis is based on the observation that, when a reconstituted basement membrane matrix, such as Matrigel, supplemented with growth factor (e.g., FGF-1), is injected subcutaneously into a host animal, endothelial cells are recruited into the matrix, forming new blood vessels over a period of several days. See, e.g., Passaniti et al., Lab. Invest., 67:519-528, 1992. By sampling the extract at different times, angiogenesis can be temporally dissected, permitting the identification of genes involved in all stages of angiogenesis, including, e.g., migration of endothelial cells into the matrix, commitment of endothelial cells to angiogenesis pathway, cell elongation and formation of sac-like spaces, and establishment of functional capillaries comprising connected, and linear structures containing red blood cells. To stabilize the growth factor and/or slow its release from the matrix, the growth factor can be bound to heparin or another stabilizing agent. The matrix can also be periodically re-infused with growth factor to enhance and extend the angiogenic process.

Other useful systems for studying angiogenesis, include, e.g., neovascularization of tumor explants (e.g., U.S. Pat. Nos. 5,192,744; 6,024,688), chicken chorioallantoic membrane (CAM) assay (e.g., Taylor and Folkman, Nature, 297:307-312, 1982; Eliceiri et al., J. Cell Biol., 140, 1255-1263, 1998), bovine capillary endothelial (BCE) cell assay (e.g., U.S. Pat. No. 6,024,688; Polverini, P. J. et al., Methods Enzymol., 198: 440-450, 1991), migration assays, HUVEC (human umbilical cord vascular endothelial cell) growth inhibition assay (e.g., U.S. Pat. No. 6,060,449).

The entire disclosure of all applications, patents and publications cited above and below, including U.S. provisional application Nos. 60/808,555, filed May 26, 2006 and 60/859,241, filed Nov. 16, 2006 and its appendix “Appendix A” are incorporated in this application by reference in their entirety.

Phase I Data:

“Phase I data on the combination of Sorafenib, Carboplatin and Paclitaxel in treating solid tumors is reported by Flaherty et al. in the document entitled “A Phase I Trial of the Oral, Multi-kinase Inhibitor Sorafenib in Combination with Carboplatin and Paclitaxel” and appended to U.S. provisional application 60/859,241, filed Nov. 16, 2006 as “Appendix A.”

What is claimed is:

1. A combination which is therapeutically effective for the treatment of mammalian cancer comprising:
(1) at least one substituted-diaryl urea of Formula I, or a polymorph, solvate, hydrate, metabolite, prodrug, diastereoisomer or pharmaceutically acceptable salt thereof, 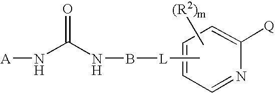
wherein
Q is —C(O)R)ₓ
Rₓ is hydroxy, C₁₋₄ alkyl, C₁₋₄ alkoxy or NRₐR_{b},
Rₐ and R_{b} are independently:
a) hydrogen;
b) C₁₋₄ alkyl, optionally substituted by
hydroxy,
C₁₋₄ alkoxy,
a heteroaryl group which is pyridine, pyrimidine, pyridazine, isoquioline or quinoline;
a heterocyclic group which is piperazine or piperidine,
amino(—NH₂) optionally substituted by one or two C₁₋₄ alkyl groups, or
phenyl, or
c) phenyl optionally substituted with
halogen, or
amino (—NH₂) optionally substituted by one or two C₁₋₄ alkyl groups, or
A is phenyl of the formula 1xx, pyridyl of the formula 1x or naphthyl of the formula 1y: 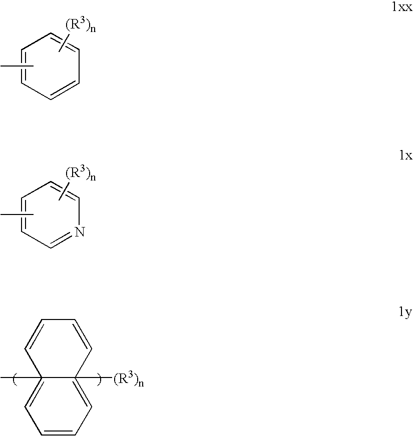
B is phenyl of formula 2a or naphthyl of formula 2b: 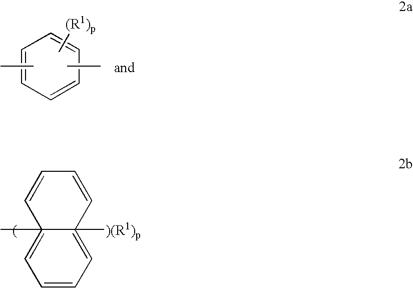
L is a bridging group which is —S— or —O—;
m is 0, 1, 2 or 3,
p is 0, 1, 2, 3, or 4,
n is 0, 1, 2, 3, 4, 5 or 6,
each R¹ is independently halogen, C₁₋₅ haloalkyl, NO₂, C(O)NR⁴R⁵, C₁₋₆ alkyl, C₁₋₆ dialkylamine, C₁₋₃ alkylamine, CN, amino, hydroxy or C₁₋₃ alkoxy;
each R² is independently C₁₋₅ alkyl, C₁₋₅ haloalkyl, C₁₋₃alkoxy, N-oxo or N-hydroxy,
each R³ is independently halogen, R⁴, OR⁴, S(O)R⁴, C(O)R⁴, C(O)NR⁴R⁵, oxo, cyano or nitro (NO₂), and
R⁴ and R⁵ are independently hydrogen, C₁₋₆ alkyl, and up to per-halogenated C₁₋₆ alkyl;
(2) at least one taxane, or a stereoisomer thereof, a precursor (prodrug) thereof or a pharmaceutically acceptable salt thereof, and
(3) at least one platinum complex antineoplastic nucleic acid binding agent or a stereoisomer thereof, a precursor (prodrug) thereof or a pharmaceutically acceptable salt thereof.

2. A combination of claim 1 which is therapeutically effective amount for the treatment of non-small cell lung cancer (NSCLC) comprising:
(1) a substituted-diaryl urea which is 4-{4-[({[4-chloro-3-(trifluoromethyl)phenyl]amino}carbonyl)amino]phenoxy}-N-methylpyridine-2-carboxamide of the formula A, or a polymorph, solvate, hydrate, metabolite, prodrug, or pharmaceutically acceptable salt thereof, 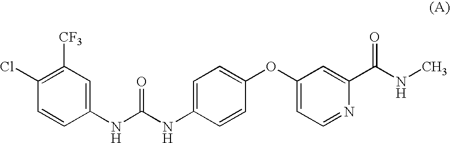
(2) at least one taxane, which is Paclitaxel (Taxol®), Docetaxel (Taxotere®), Abraxane™ or a pharmaceutically acceptable salt thereof, and
(3) at least one platinum complex antineoplastic nucleic acid binding agent which is carboplatin (Paraplatin®), oxaplatin (Eloxatin®), cisplatin (Platinol®), or a pharmaceutically acceptable salt thereof.

3. A combination of claim 2 which is therapeutically effective for the treatment of non-small cell lung cancer (NSCLC) comprising:
(a) a pharmaceutically acceptable salt of a substituted-diaryl urea which is the tosylate salt of N-(4-chloro-3-(trifluoromethyl)phenyl-N′-(4-(2-(N-methylcarbamoyl)-4-pyridyloxy)phenyl)urea;
(b) paclitaxel, or a pharmaceutically acceptable salt thereof, and
(c) carboplatin, or a pharmaceutically acceptable salt thereof.

4. A combination as in claim 3 adapted for administration of components (1), (2) and (3) to a patient in need thereof either
(a) in the same formulation,
(b) in separate formulations using the same administration route, or
(c) in separate formulations using different administration routes.

5. A combination as in claim 4 adapted for administration of components (1), (2) and (3) to a patient in need thereof by oral delivery and/or by intravenous injection, infusion, intramuscular, subcutaneous or parenteral route of administration.

6. A combination as in claim 5 adapted for administration to of the following agents to a patient in need thereof:
(1) at least one substituted-diaryl urea, or a polymorph, solvate, hydrate, metabolite, prodrug, diastereoisomer or pharmaceutically acceptable salt thereof, once or more per day for up to 28 consecutive days,
with either concurrent or intermittent administration of
(2) at least one taxane, or a stereoisomer thereof, a precursor (prodrug) thereof or a pharmaceutically acceptable salt thereof, and
(3) at least one platinum complex antineoplastic nucleic acid binding agent or a stereoisomer thereof, a precursor (prodrug) thereof or a pharmaceutically acceptable salt thereof, over the same period.

7. A combination as in claim 6 adapted for administration of the following agents to a patient in need thereof:
(1) at least one substituted diaryl urea compound, or a polymorph, solvate, hydrate, metabolite, prodrug, diastereoisomer or pharmaceutically acceptable salt thereof, at a dosage within the range from about 0.1 to about 300 mg/kg of total body weight,
(2) at least one taxane or a stereoisomer thereof, a precursor (prodrug) thereof or a pharmaceutically acceptable salt thereof, at a dosage range from about 10-300 mg/m² of patient surface area, and
(3) at least one platinum complex or a stereoisomer thereof, a precursor (prodrug) thereof or a pharmaceutically acceptable salt thereof, within the range from about 100-500 mg/m² of patient surface area.

8. A combination of claim 1 wherein
R¹ of Formula I is chlorine or fluorine;
R² of Formula I is methyl or trifluoromethyl;
R³ of Formula I is trifluoromethyl, methyl, ethyl, propyl, butyl, isopropyl, tert-butyl, chlorine, fluorine, bromine, cyano, methoxy, acetyl, trifluoromethanesulfonyl, trifluoromethoxy, or trifluoromethylthio;
A of Formula I is 3-tert butyl phenyl, 5-tert butyl-2-methoxyphenyl, 5-(trifluoromethyl)-2 phenyl, 3-(trifluoromethyl)-4 chlorophenyl, 3-trifluoromethyl)-4-bromophenyl or 5-(trifluoromethyl)-4-chloro-2 methoxyphenyl; and
B of Formula I is:

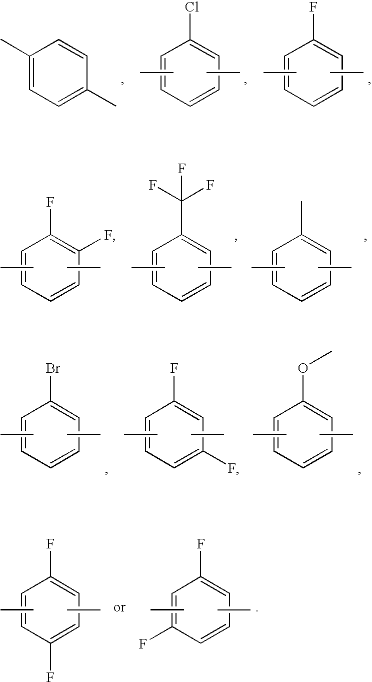

9. A combination of claim 1 wherein
R³ of Formula I is chlorine, trifluoromethyl, tert-butyl or methoxy and B is phenylene, fluoro substituted phenylene or difluoro substituted phenylene.

10. A combination of claim 1, where the substituted-diaryl urea of Formula I has the structure of Formula X 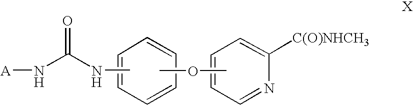
where A is as defined for Formula I in claim 1.

11. A combination of claim 3, where at least 80% of the tosylate salt of N-(4-chloro-3-(trifluoromethyl)phenyl-N′-(4-(2-(N-methylcarbamoyl)-4-pyridyloxy)phenyl)urea exists as in the stable polymorph I.

12. A kit for treating non-small cell lung cancer (NSCLC), colon, prostate, leukemia, hepatocellular, renal, head and neck, glioma, lung, pancreatic, and ovarian cancers comprising:
(1) at least one substituted-diaryl urea of Formula I, or a polymorph, solvate, hydrate, metabolite, prodrug, diastereoisomer or pharmaceutically acceptable salt thereof, 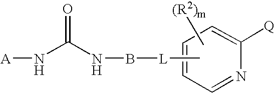
wherein
Q is —C(O)Rₓ
Rₓ is hydroxy, C₁₋₄ alkyl, C₁₋₄ alkoxy or NRₐR_{b},
Rₐ and R_{b} are independently:
a) hydrogen;
b) C₁₋₄ alkyl, optionally substituted by
hydroxy,
C₁₋₄ alkoxy,
a heteroaryl group which is pyridine, pyrimidine, pyridazine, isoquioline or quinoline;
a heterocyclic group which is piperazine or piperidine,
amino(—NH₂) optionally substituted by one or two C₁₋₄ alkyl groups, or
phenyl, or
c) phenyl optionally substituted with
halogen, or
amino (—NH₂) optionally substituted by one or two C₁₋₄ alkyl groups, or
A is phenyl of the formula 1xx, pyridyl of the formula 1x or naphthyl of the formula 1y: 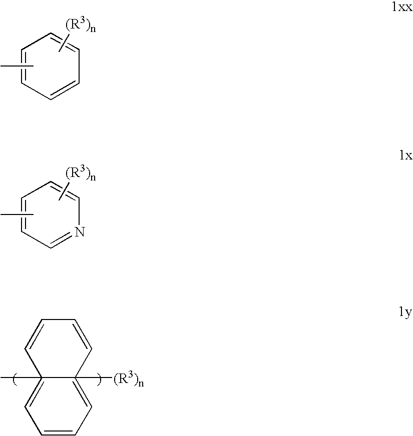
B is phenyl of formula 2a or naphthyl of formula 2b: 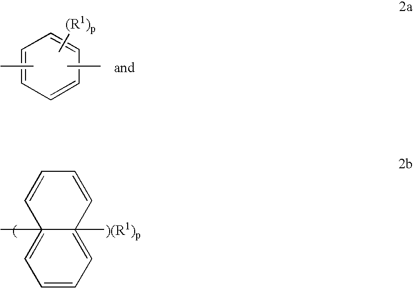
L is a bridging group which is —S— or —O—;
m is 0, 1, 2 or 3,
p is 0, 1, 2, 3, or 4,
n is 0, 1, 2, 3, 4, 5 or 6,
each R¹ is independently halogen, C₁₋₅ haloalkyl, NO₂, C(O)NR⁴R⁵, C₁₋₆ alkyl, C₁₋₆ dialkylamine, C₁₋₃ alkylamine, CN, amino, hydroxy or C₁₋₃ alkoxy;
each R² is independently C₁₋₅ alkyl, C₁₋₅ haloalkyl, C₁₋₃ alkoxy, N-oxo or N-hydroxy,
each R³ is independently halogen, R⁴, OR⁴, S(O)R⁴, C(O)R⁴, C(O)NR⁴R⁵, oxo, cyano or nitro (NO₂), and
R⁴ and R⁵ are independently hydrogen, C₁₋₆ alkyl, and up to per-halogenated C₁₋₆ alkyl;
(2) at least one taxane, or a stereoisomer thereof, a precursor (prodrug) thereof or a pharmaceutically acceptable salt thereof, and
(3) at least one platinum complex antineoplastic nucleic acid binding agent or a stereoisomer thereof, a precursor (prodrug) thereof or a pharmaceutically acceptable salt thereof.

13. A kit as in claim 12 comprising separate doses, in separate containers, of therapeutically effective amounts of:
(1) a substituted-diaryl urea of Formula I which is 4-{4-[({[4-chloro-3-(trifluoromethyl)phenyl]amino}carbonyl)amino]phenoxy}-N-methylpyridine-2-carboxamide of the formula A, or a polymorph, solvate, hydrate, metabolite, prodrug, or pharmaceutically acceptable salt thereof, 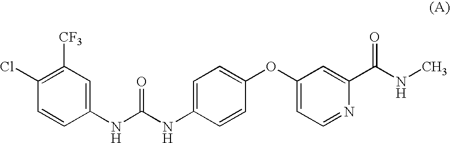
(2) at least one taxane, which is Paclitaxel (Taxol®), Docetaxel (Taxotere®), Abraxane™ or a stereoisomer thereof, a precursor (prodrug) thereof or a pharmaceutically acceptable salt thereof, and
(3) at least one platinum complex antineoplastic nucleic acid binding agent which is carboplatin (Paraplatin®), oxaplatin (Eloxatin®), cisplatin (Platinol®), or a stereoisomer thereof, a precursor (prodrug) thereof or a pharmaceutically acceptable salt thereof.

14. A kit as in claim 12 comprising separate doses, in separate containers, of therapeutically effective amounts of:
(a) a pharmaceutically acceptable salt of a substituted-diaryl urea of Formula I which is the tosylate salt of N-(4-chloro-3-(trifluoromethyl)phenyl-N′-(4-(2-N-methylcarbamoyl)-4-pyridyloxy)phenyl)urea;
(b) paclitaxel, or a stereoisomer thereof, a precursor (prodrug) thereof or a pharmaceutically acceptable salt thereof, and
(c) carboplatin, or a stereoisomer thereof, a precursor (prodrug) thereof or a pharmaceutically acceptable salt thereof.

15. A kit of claim 14 for the treatment of non-small cell lung carcinoma.

16. A kit as in claim 14 wherein the components are within a single pharmaceutical formulation in a single container.

17. A kit as in claim 14 wherein the components are within separate formulations, in separate containers.

18. A kit of claim 12 wherein
R¹ of Formula I is chlorine or fluorine;
R² of Formula I is methyl or trifluoromethyl;
R³ of Formula I is trifluoromethyl, methyl, ethyl, propyl, butyl, isopropyl, tert-butyl, chlorine, fluorine, bromine, cyano, methoxy, acetyl, trifluoromethanesulfonyl, trifluoromethoxy, or trifluoromethylthio;
A of Formula I is 3-tert butyl phenyl, 5-tert butyl-2-methoxyphenyl, 5-(trifluoromethyl)-2 phenyl, 3-(trifluoromethyl)-4 chlorophenyl, 3-trifluoromethyl)-4-bromophenyl or 5-(trifluoromethyl)-4-chloro-2 methoxyphenyl; and
B of Formula I is:

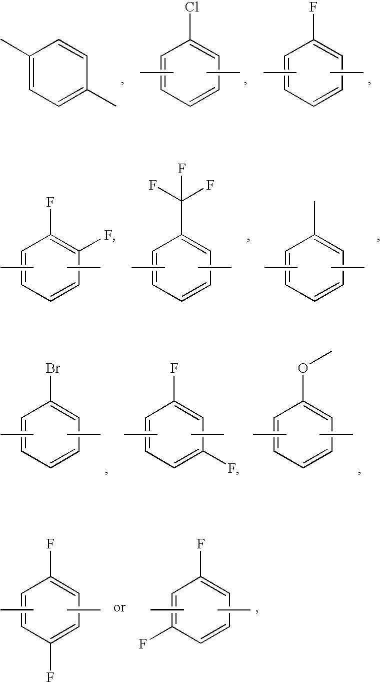

19. A kit of claim 12 wherein
R³ of Formula I is chlorine or trifluoromethyl and B is phenylene, fluoro substituted phenylene or difluoro substituted phenylene.

20. A kit of claim 12 where the substituted-diaryl urea of Formula I has the structure of Formula X 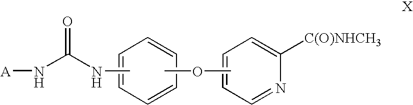
where A is as defined for Formula I.

21. A kit of claim 14 where at least 80% of the tosylate salt of N-(4-chloro-3-(trifluoromethyl)phenyl-N′-(4-(2-(N-methylcarbamoyl)-4-pyridyloxy)phenyl)urea exists as in the stable polymorph I.

22. A kit as in claim 12, wherein
(1) the dosage of the substituted-diaryl urea or a polymorph, solvate, hydrate, metabolite, prodrug, or pharmaceutically acceptable salt thereof is 50 to 600 mg,
(2) the dosage of the taxane or stereoisomer, precursor (prodrug) or pharmaceutically acceptable salt thereof is from about 30 to 200 mg/m² and
(3) the dosage for the platinum complex antineoplastic nucleic acid binding agent or stereoisomer, precursor (prodrug) or pharmaceutically acceptable salt thereof is 100 to 500 mg/m².

23. A pharmaceutical composition for the treatment of mammalian cancer comprising a combination as in claim 1 and at least one pharmaceutically acceptable carrier.

24. A pharmaceutical composition for the treatment of non-small cell lung cancer comprising a combination as in claim 1 and at least one pharmaceutically acceptable carrier.

25. A method of using a combination of claim 1 in the manufacture of a medicament for the treatment of mammalian cancer.

26. A method of using a combination of claim 1 in the manufacture of a medicament for the treatment of non-small cell lung cancer.

27. A method of using the tosylate salt of N-(4-chloro-3-(trifluoromethyl)phenyl-N′-(4-(2-(N-methylcarbamoyl)-4-pyridyloxy)phenyl)urea in the manufacture of a kit of claim 14 for the treatment of non-small cell lung cancer (NSCLC), colon, prostate, leukemia, hepatocellular, renal, head and neck, glioma, lung, pancreatic, and ovarian cancers.

28. A method of using the tosylate salt of N-(4-chloro-3-(trifluoromethyl)phenyl-N′-(4-(2-N-methylcarbamoyl)-4-pyridyloxy)phenyl)urea in the manufacture of a kit of claim 14 for the treatment of non-small cell lung cancer (NSCLC).

29. A combination as in claim 1, wherein the mammalian cancer to be treated is non-small cell lung cancer and cancer of the colon, pancreas, prostate, liver, kidney, lung, head and neck, pancreas and ovary.

30. A combination as in claim 1, wherein the mammalian cancer to be treated is
(a) small-cell lung cancer, non-small-cell lung cancer, bronchial adenoma, pleuropulmonary blastoma;
(b) brain stem and hypophtalmic glioma, cerebellar and cerebral astrocytoma, medulloblastoma, ependymoma, and neuroectodermal, pineal tumor;
(c) endometrial cancer, cervical cancer, ovarian cancer, vaginal cancer, vulvar cancer, sarcoma of the uterus bone;
(d) anal cancer, colon cancer, colorectal cancer, esophageal cancer, gallbladder cancer, gastric cancer, pancreatic cancer, rectal cancer, small intestine cancer, salivary gland cancer;
(e) hepatocellular carcinoma, cholangiocarcinoma, mixed hepatocellular cholangiocarcinoma;
(f) bladder cancer, penile cancer, kidney cancer, kidney neoplasm, renal pelvis cancer, ureter cancer, urethral cancer;
(g) squamous cell carcinoma, Kaposi's sarcoma, malignant melanoma, Merkel cell skin cancer, non-melanoma skin cancer;
(h) intraocular melanoma, retinoblastoma;
(i) laryngeal, hypopharyngeal, nasopharyngeal, oropharyngeal cancers, lip and oral cavity cancer;
(j) testicular cancer, prostate cancer;
(k) AIDS-related lymphoma, non-Hodgkin's lymphoma, cutaneous T-cell lymphoma, Hodgkin's disease, lymphoma of the central nervous system;
(l) glioblastoma, hematologic malignancies, Lhermitte-Duclose disease, malignant glioma, multiple myeloma, myeloid metaplasia, myeloplastic syndromes;
(m) sarcoma of the soft tissue, osteosarcoma, malignant fibrous histiocytoma, lymphosarcoma, rhabdomyosarcoma, leimyosarcoma, liposarcoma;
(n) acute myeloid leukemia, acute lymphoblastic leukemia, chronic lymphocytic leukemia, chronic myelogenous leukemia, and hairy cell leukemia.

31. A pharmaceutical composition of claim 23 wherein the mammalian cancer to be treated is non-small cell lung cancer, cancer of the colon, pancreas, prostate, liver, kidney, lung, head and neck, pancreas and ovary.

32. A method of treating a hyperproliferative disease in a patient comprising administering to said patient an effective amount of a combination of claim 1.

33. A method of claim 32 wherein said hyperproliferative disorder is brain tumor, breast cancer, bone sarcoma, bronchial premalignancy, endometrial cancer, glioblastoma, hematologic malignancies, hepatocellular carcinoma, Hodgkin's disease, kidney neoplasms, leukemia, leimyosarcoma, liposarcoma, lymphoma, Lhermitte-Duclose disease, malignant glioma, melanoma, malignant melanoma, metastases, multiple myeloma, myeloid metaplasia, myeloplastic syndromes, non-small cell lung cancer, pancreatic cancer, prostate cancer, renal cell carcinoma, rhabdomyosarcoma, soft tissue sarcoma, squamous epithelial carcinoma of the skin or a combination thereof.

34. A method of claim 32 wherein said hyperproliferative disorder is non-small cell lung cancer (NSCLC).

35. A method of claim 32 wherein said combination comprises
(a) a diaryl urea which is administered to said patient at a dosage range from about 0.1-300 mg/kg of total body weight,
(b) a taxane which is administered to said patient at a dosage range from about 10-300 mg/m² of patient surface area and
(c) a platinum complex which is administered to said patient at a dosage range from about 100-500 mg/m² of patient surface area.

36. A method of claim 35 wherein said combination is administered to said patient in intravenous, intramuscular, subcutaneous, or parenteral route of administration.

37. A method of claim 36 wherein (a), (b), and (c) are administered sequentially.

38. A method of treating a hyperproliferative disease in a patient comprising administering to said patient an effective amount of a pharmaceutical composition of claim 3.

39. A method of claim 38 wherein said hyperproliferative disorder is brain tumor, breast cancer, bone sarcoma, bronchial premalignancy, endometrial cancer, glioblastoma, hematologic malignancies, hepatocellular carcinoma, Hodgkin's disease, kidney neoplasms, leukemia, leimyosarcoma, liposarcoma, lymphoma, Lhermitte-Duclose disease, malignant glioma, melanoma, malignant melanoma, metastases, multiple myeloma, myeloid metaplasia, myeloplastic syndromes, non-small cell lung cancer, pancreatic cancer, prostate cancer, renal cell carcinoma, rhabdomyosarcoma, soft tissue sarcoma, squamous epithelial carcinoma of the skin or a combination thereof.

40. A method of claim 38 wherein said hyperproliferative disorder is non-small cell lung cancer (NSCLC).

41. A method of claim 7 wherein the effective amount of said combination in said pharmaceutical comprises
component (a) administered to said patient at a dosage range from about 0.1-300 mg/kg of total body weight,
component (b) administered to said patient at a dosage range from about 10-300 mg/m² of patient surface area and
component (c) administered to said patient at a dosage range from about 100-500 mg/m² of patient surface area.

42. A method of claim 41 wherein said composition is administered to said patient in intravenous, intramuscular, subcutaneous, or parenteral route of administration.

43. A method of claim 40 wherein the non-small cell lung cancer (NSCLC) is squamous cell (epidermoid) carcinoma, adenocarcinoma, large cell carcinoma, adenosquamous carcinoma, and undifferentiated carcinoma.

44. A method of claim 40 wherein the non-small cell lung cancer (NSCLC) is surgically resectable or advanced.

45. A method of claim 40 wherein the non-small cell lung cancer (NSCLC) is metastatic or local.
